(19) United States
(12) Patent Application Publication
(10) Pub. No.: US 2025/0001109 A1
(43) Pub. Date: Jan. 2, 2025

(54) ENHANCED WEARABLE SENSING

(71) Applicant: ResMed Digital Health Inc., San Diego, CA (US)

(72) Inventors: Redmond SHOULDICE, Dublin (IE); Michael WREN, Dublin (IE); Stephen MCMAHON, Dublin (IE); Kieran GRENNAN, Dublin (IE)

(21) Appl. No.: 18/709,064

(22) PCT Filed: Nov. 4, 2022
(86) PCT No.: PCT/IB2022/060625

§ 371 Date: May 10, 2024

Related U.S. Application Data
(60) Provisional application No. 63/277,828, filed on Nov. 10, 2021.

(51) Int. Cl. A61M 16/00; A61M 16/06

(52) U.S. Cl. CPC A61M 16/024; A61M 16/06; A61M 2205/3303; A61M 2205/3375; A61M 2205/3561; A61M 2209/086

(57) ABSTRACT

A wearable device can automatically switch between modes of collecting sensor data when a docking event is detected between the wearable device and a docking device. In a first mode (e.g., when undocked), data can be collected using a first sensor configuration (e.g., a first set of sensors operating using a first set of sensing parameters). In a second mode (e.g., when docked), data can be collected using a second sensor configuration, which can include the use of one or more different sensors and/or the use of one or more different sensing parameters. The first mode may prioritize battery life, whereas the second mode may prioritize sensor data fidelity, such as by increasing sampling rates, using different sensors, and the like. Sensor data from the first and second modes can be used individually (e.g., to calibrate the other) and/or together (e.g., to determine physiological parameters).

### CROSS-REFERENCE TO RELATED APPLICATIONS

This application claims the benefit of, and priority to, U.S. Provisional Patent Application No. 63/277,828 filed on Nov. 10, 2021, which is hereby incorporated by reference herein in its entirety.

### TECHNICAL FIELD

The present disclosure relates generally to wearable devices, and more particularly, to systems and methods for providing intelligent monitoring of a user also when the wearable device is in an unworn configuration.

### BACKGROUND

Many individuals suffer from sleep-related and/or respiratory-related disorders such as, for example, Periodic Limb Movement Disorder (PLMD), Restless Leg Syndrome (RLS), Sleep-Disordered Breathing (SDB) such as Obstructive Sleep Apnea (OSA), Central Sleep Apnea (CSA), other types of apneas such as mixed apneas and hypopneas, Respiratory Effort Related Arousal (RERA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), shift work sleep disorder, non-24-hour sleep-wake disorder, hypertension, diabetes, stroke, insomnia, and chest wall disorders.

Data is often collected to facilitate diagnosis and treatment of such sleep-related and/or respiratory-related disorders. Often, high-quality data collection requires visits to a sleep clinic for data collection or the use of specialized monitoring equipment in one's own home. While such techniques can provide useful data that facilitates diagnosing and treating sleep-related and/or respiratory-related disorders, the bar to entry is very high, which can make such techniques unsuitable for many individuals who are or are not diagnosed with a sleep-related and/or respiratory-related disorder.

Wearable devices can be used on a daily basis to collect data that may be useful to diagnosing and/or treating physiological conditions/disorders, such as sleep-related and/or respiratory-related disorders, among other uses. Such other uses include monitoring physiological parameters, such as heart rate, respiration rate, body temperature, etc. Because of the small size requirements of wearable devices, the types of sensors used and the sizes of batteries used are limited. Thus, wearable devices that are small enough to be conveniently worn by a user are generally limited in the quality and quantity of data they can obtain. Once the wearable device's battery becomes depleted, the user must recharge or replace the wearable device's battery before continuing with data collection. For some multi-purpose devices, such as smartwatches, which also operate as a timepiece and often provide additional features, the most common time to recharge such devices is while the user is asleep (e.g., when the user is not intending to actively use the various features of the device). Thus, common use of many wearable devices leave large breaks in collected data. For certain use cases, such as the diagnosis and treatment of sleep-related and/or respiratory-related disorders, the most common timing of these large breaks in collected data fall at extremely inopportune times, such as while the user is sleeping (e.g., to collect sleep-related data).

The present disclosure is directed to solving these and other problems.

### SUMMARY

According to some implementations of the present disclosure, a method includes operating a wearable device in a first mode. The wearable device has one or more sensors. Operating the wearable device in the first mode includes receiving first sensor data from at least one of the one or more sensors of the wearable device while the wearable device is being worn by a user. The method further includes detecting a docking event associated with coupling the wearable device to a docking device. The wearable device receives power from the docking device when the wearable device is coupled with the docking device. The method further includes automatically operating the wearable device in a second mode in response to detecting the docking event. Operating the wearable device in the second mode includes receiving second sensor data. The method can further include determining a physiological parameter associated with the user based at least in part on the first sensor data and the second sensor data. The physiological parameter can be usable to facilitate diagnosis and/or treatment of a disorder, such as a sleep-related and/or respiratory-related disorder.

According to some implementations of the present disclosure, a system includes a memory and a control system. The memory stores machine-readable instructions. The control system includes one or more processors configured to execute the machine-readable instructions to operating a wearable device in a first mode. The wearable device has one or more sensors. Operating the wearable device in the first mode includes receiving first sensor data from at least one of the one or more sensors of the wearable device while the wearable device is being worn by a user. The control system is further configured to detect a docking event associated with coupling the wearable device to a docking device. The wearable device receives power from the docking device when the wearable device is coupled with the docking device. The control system is further configured to automatically operate the wearable device in a second mode in response to detecting the docking event. Operating the wearable device in the second mode includes receiving second sensor data. The control system can be further configured to determine a physiological parameter associated with the user based at least in part on the first sensor data and the second sensor data. The physiological parameter can be usable to facilitate diagnosis and/or treatment of a disorder, such as a sleep-related and/or respiratory-related disorder.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system, according to some implementations of the present disclosure.

FIG. 2 is a perspective view of at least a portion of the system of FIG. 1, a user, and a bed partner, according to some implementations of the present disclosure.

FIG. 3 illustrates an exemplary timeline for a sleep session, according to some implementations of the present disclosure.

FIG. 4 illustrates an exemplary hypnogram associated with the sleep session of FIG. 3, according to some implementations of the present disclosure.

FIG. 5 is a schematic diagram depicting a wearable device operating in a first mode, according to certain aspects of the present disclosure.

FIG. 6 is a schematic diagram depicting a wearable device operating in a second mode while docked with a mains-powered docking device, according to certain aspects of the present disclosure.

FIG. 7 is a schematic diagram depicting a wearable device operating in a second mode while docked with a battery-powered docking device, according to certain aspects of the present disclosure.

FIG. 8 is a chart depicting sensor configurations before and after a docking event, according to certain aspects of the present disclosure.

FIG. 9 is a flowchart depicting a process for automatically switching modes of a wearable device in response to detecting a docking event, according to certain aspects of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Systems and methods are disclosed for using a wearable device to collect sensor data and automatically switching between modes of collecting sensor data upon detection of a docking event between the wearable device and a docking device. Data collection in a first mode (e.g., when the wearable device is undocked) can be collected using a first sensor configuration (e.g., a first set of sensors operating using a first set of sensing parameters), whereas data collection in a second mode (e.g., when the wearable device is docked) can be collected using a different, second sensor configuration, which can include the use of one or more different sensors and/or the use of one or more different sensing parameters. For example, the first mode may prioritize battery life and the use of certain sensors on the wearable device, whereas the second mode may prioritize sensor data fidelity, such as by increasing sampling rates, using different sensors, and the like. The sensor data collected in the first mode and the sensor data collected in the second mode can be used together to determine physiological parameters and/or can be used individually to calibrate the other, among other uses.

Certain aspects and features of the present disclosure are especially useful for collecting physiological data, such as sleep-related physiological data associated with a sleep session of a user. Such data can be especially useful to facilitate diagnosing and/or treating sleep-related and/or respiratory-related disorders.

Many individuals suffer from sleep-related and/or respiratory disorders. Examples of sleep-related and/or respiratory disorders include Periodic Limb Movement Disorder (PLMD), Restless Leg Syndrome (RLS), Sleep-Disordered Breathing (SDB) such as Obstructive Sleep Apnea (OSA). Central Sleep Apnea (CSA), and other types of apneas such as mixed apneas and hypopneas, Respiratory Effort Related Arousal (RERA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), shift work sleep disorder, non-24-hour sleep-wake disorder, hypertension, diabetes, stroke, insomnia, parainsomnia, and chest wall disorders.

Obstructive Sleep Apnea (OSA) is a form of Sleep Disordered Breathing (SDB), and is characterized by events including occlusion or obstruction of the upper air passage during sleep resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as Central Sleep Apnea). Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events must fulfil both of the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040 and U.S. Pat. No. 9,358,353, assigned to ResMed Ltd., the disclosure of each of which is hereby incorporated by reference herein in their entireties.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

Rapid eye movement behavior disorder (RBD) is characterized by a lack of muscle atonia during REM sleep, and in more severe cases, movement and speech produced by an individual during REM sleep stages. RBD can sometimes be accompanied by dream enactment behavior (DEB), where the individual acts out dreams they may be having, sometimes resulting in injuries to themselves or their partners. RBD is often a precursor to a subclass of neuro-degenerative disorders, such as Parkinson's disease, Lewis Body Dementia, and Multiple System Atrophy. Typically, RBD is diagnosed in a sleep laboratory via poly somnography. This process can be expensive, and often occurs late in the evolution process of the disease, when mitigating therapies are difficult to adopt and/or less effective. Monitoring an individual during sleep in a home environment or other common sleeping environment can beneficially be used to identify whether the individual is suffering from RBD or DEB.

Shift work sleep disorder is a circadian rhythm sleep disorder characterized by a circadian misalignment related to a work schedule that overlaps with a traditional sleep-wake cycle. This disorder often presents as insomnia when attempting to sleep and/or excessive sleepiness while working for an individual engaging in shift work. Shift work can involve working nights (e.g., after 7 pm), working early mornings (e.g., before 6 am), and working rotating shifts. Left untreated, shift work sleep disorder can result in complications ranging from light to serious, including mood problems, poor work performance, higher risk of accident, and others.

Non-24-hour sleep-wake disorder (N24SWD), formally known as free-running rhythm disorder or hypernychthemeral syndrome, is a circadian rhythm sleep disorder in which the body clock becomes desynchronized from the environment. An individual suffering from N24SWD will have a circadian rhythm that is shorter or longer than 24 hours, which causes sleep and wake times to be pushed progressively earlier or later. Over time, the circadian rhythm can become desynchronized from regular daylight hours, which can cause problematic fluctuations in mood, appetite, and alertness. Left untreated, N24SWD can result in further health consequences and other complications.

Many individuals suffer from insomnia, a condition which is generally characterized by a dissatisfaction with sleep quality or duration (e.g., difficulty initiating sleep, frequent or prolonged awakenings after initially falling asleep, and an early awakening with an inability to return to sleep). It is estimated that over 2.6 billion people worldwide experience some form of insomnia, and over 750 million people worldwide suffer from a diagnosed insomnia disorder. In the United States, insomnia causes an estimated gross economic burden of $107.5 billion per year, and accounts for 13.6% of all days out of role and 4.6% of injuries requiring medical attention. Recent research also shows that insomnia is the second most prevalent mental disorder, and that insomnia is a primary risk factor for depression.

Nocturnal insomnia symptoms generally include, for example, reduced sleep quality, reduced sleep duration, sleep-onset insomnia, sleep-maintenance insomnia, late insomnia, mixed insomnia, and/or paradoxical insomnia. Sleep-onset insomnia is characterized by difficulty initiating sleep at bedtime. Sleep-maintenance insomnia is characterized by frequent and/or prolonged awakenings during the night after initially falling asleep. Late insomnia is characterized by an early morning awakening (e.g., prior to a target or desired wakeup time) with the inability to go back to sleep. Comorbid insomnia refers to a type of insomnia where the insomnia symptoms are caused at least in part by a symptom or complication of another physical or mental condition (e.g., anxiety, depression, medical conditions, and/or medication usage). Mixed insomnia refers to a combination of attributes of other types of insomnia (e.g., a combination of sleep-onset, sleep-maintenance, and late insomnia symptoms). Paradoxical insomnia refers to a disconnect or disparity between the user's perceived sleep quality and the user's actual sleep quality.

Diurnal (e.g., daytime) insomnia symptoms include, for example, fatigue, reduced energy, impaired cognition (e.g., attention, concentration, and/or memory), difficulty functioning in academic or occupational settings, and/or mood disturbances. These symptoms can lead to psychological complications such as, for example, lower mental (and/or physical) performance, decreased reaction time, increased risk of depression, and/or increased risk of anxiety disorders. Insomnia symptoms can also lead to physiological complications such as, for example, poor immune system function, high blood pressure, increased risk of heart disease, increased risk of diabetes, weight gain, and/or obesity.

Co-morbid Insomnia and Sleep Apnea (COMISA) refers to a type of insomnia where the subject experiences both insomnia and obstructive sleep apnea (OSA). OSA can be measured based on an Apnea-Hypopnea Index (AHI) and/or oxygen desaturation levels. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild OSA. An AHI that is greater than or equal to 15, but less than 30 is considered indicative of moderate OSA. An AHI that is greater than or equal to 30 is considered indicative of severe OSA. In children, an AHI that is greater than 1 is considered abnormal.

Insomnia can also be categorized based on its duration. For example, insomnia symptoms are considered acute or transient if they occur for less than 3 months. Conversely, insomnia symptoms are considered chronic or persistent if they occur for 3 months or more, for example. Persistent/chronic insomnia symptoms often require a different treatment path than acute/transient insomnia symptoms.

Known risk factors for insomnia include gender (e.g., insomnia is more common in females than males), family history, and stress exposure (e.g., severe and chronic life events). Age is a potential risk factor for insomnia. For example, sleep-onset insomnia is more common in young adults, while sleep-maintenance insomnia is more common in middle-aged and older adults. Other potential risk factors for insomnia include race, geography (e.g., living in geographic areas with longer winters), altitude, and/or other sociodemographic factors (e.g. socioeconomic status, employment, educational attainment, self-rated health, etc.).

Mechanisms of insomnia include predisposing factors, precipitating factors, and perpetuating factors. Predisposing factors include hyperarousal, which is characterized by increased physiological arousal during sleep and wakefulness. Measures of hyperarousal include, for example, increased levels of cortisol, increased activity of the autonomic nervous system (e.g., as indicated by increase resting heart rate and/or altered heart rate), increased brain activity (e.g., increased EEG frequencies during sleep and/or increased number of arousals during REM sleep), increased metabolic rate, increased body temperature and/or increased activity in the pituitary-adrenal axis. Precipitating factors include stressful life events (e.g., related to employment or education, relationships, etc.) Perpetuating factors include excessive worrying about sleep loss and the resulting consequences, which may maintain insomnia symptoms even after the precipitating factor has been removed.

Conventionally, diagnosing or screening insomnia (including identifying a type or insomnia and/or specific symptoms) involves a series of steps. Often, the screening process begins with a subjective complaint from a patient (e.g., they cannot fall or stay sleep).

Next, the clinician evaluates the subjective complaint using a checklist including insomnia symptoms, factors that influence insomnia symptoms, health factors, and social factors. Insomnia symptoms can include, for example, age of onset, precipitating event(s), onset time, current symptoms (e.g., sleep-onset, sleep-maintenance, late insomnia), frequency of symptoms (e.g., every night, episodic, specific nights, situation specific, or seasonal variation), course since onset of symptoms (e.g., change in severity and/or relative emergence of symptoms), and/or perceived daytime consequences. Factors that influence insomnia symptoms include, for example, past and current treatments (including their efficacy), factors that improve or ameliorate symptoms, factors that exacerbate insomnia (e.g., stress or schedule changes), factors that maintain insomnia including behavioral factors (e.g., going to bed too early, getting extra sleep on weekends, drinking alcohol, etc.) and cognitive factors (e.g., unhelpful beliefs about sleep, worry about consequences of insomnia, fear of poor sleep, etc.). Health factors include medical disorders and symptoms, conditions that interfere with sleep (e.g., pain, discomfort, treatments), and pharmacological considerations (e.g., alerting and sedating effects of medications). Social factors include work schedules that are incompatible with sleep, arriving home late without time to wind down, family and social responsibilities at night (e.g., taking care of children or elderly), stressful life events (e.g., past stressful events may be precipitants and current stressful events may be perpetuators), and/or sleeping with pets.

After the clinician completes the checklist and evaluates the insomnia symptoms, factors that influence the symptoms, health factors, and/or social factors, the patient is often directed to create a daily sleep diary and/or fill out a questionnaire (e.g., Insomnia Severity Index or Pittsburgh Sleep Quality Index). Thus, this conventional approach to insomnia screening and diagnosis is susceptible to error(s) because it relies on subjective complaints rather than objective sleep assessment. There may be a disconnect between patient's subjective complaint(s) and the actual sleep due to sleep state misperception (paradoxical insomnia).

In addition, the conventional approach to insomnia diagnosis does not rule out other sleep-related disorders such as, for example, Periodic Limb Movement Disorder (PLMD), Restless Leg Syndrome (RLS), Sleep-Disordered Breathing (SDB), Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders. These other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping. While these other sleep-related disorders may have similar symptoms as insomnia, distinguishing these other sleep-related disorders from insomnia is useful for tailoring an effective treatment plan distinguishing characteristics that may call for different treatments. For example, fatigue is generally a feature of insomnia, whereas excessive daytime sleepiness is a characteristic feature of other disorders (e.g., PLMD) and reflects a physiological propensity to fall asleep unintentionally.

Once diagnosed, insomnia can be managed or treated using a variety of techniques or providing recommendations to the patient. A plan of therapy used to treat insomnia, or other sleep-related disorders, can be known as a sleep therapy plan. For insomnia, the patient might be encouraged or recommended to generally practice healthy sleep habits (e.g., plenty of exercise and daytime activity, have a routine, no bed during the day, eat dinner early, relax before bedtime, avoid caffeine in the afternoon, avoid alcohol, make bedroom comfortable, remove bedroom distractions, get out of bed if not sleepy, try to wake up at the same time each day regardless of bed time) or discouraged from certain habits (e.g., do not work in bed, do not go to bed too early, do not go to bed if not tired). The patient can additionally or alternatively be treated using sleep medicine and medical therapy such as prescription sleep aids, over-the-counter sleep aids, and/or at-home herbal remedies.

The patient can also be treated using cognitive behavior therapy (CBT) or cognitive behavior therapy for insomnia (CBT-I), which is a type of sleep therapy plan that generally includes sleep hygiene education, relaxation therapy, stimulus control, sleep restriction, and sleep management tools and devices. Sleep restriction is a method designed to limit time in bed (the sleep window or duration) to actual sleep, strengthening the homeostatic sleep drive. The sleep window can be gradually increased over a period of days or weeks until the patient achieves an optimal sleep duration. Stimulus control includes providing the patient a set of instructions designed to reinforce the association between the bed and bedroom with sleep and to reestablish a consistent sleep-wake schedule (e.g., go to bed only when sleepy, get out of bed when unable to sleep, use the bed for sleep only (e.g., no reading or watching TV), wake up at the same time each morning, no napping, etc.) Relaxation training includes clinical procedures aimed at reducing autonomic arousal, muscle tension, and intrusive thoughts that interfere with sleep (e.g., using progressive muscle relaxation). Cognitive therapy is a psychological approach designed to reduce excessive worrying about sleep and reframe unhelpful beliefs about insomnia and its daytime consequences (e.g., using Socratic question, behavioral experiences, and paradoxical intention techniques). Sleep hygiene education includes general guidelines about health practices (e.g., diet, exercise, substance use) and environmental factors (e.g., light, noise, excessive temperature) that may interfere with sleep. Mindfulness-based interventions can include, for example, meditation.

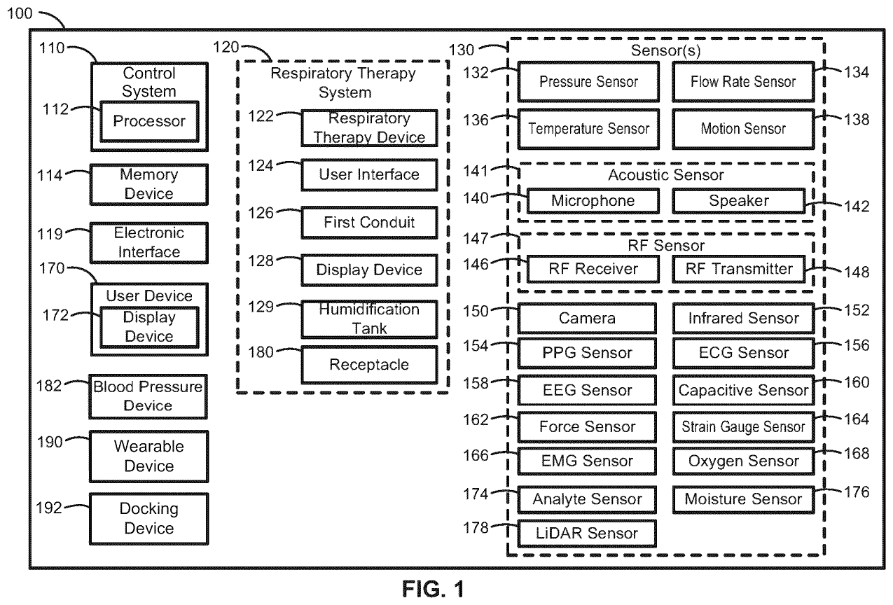

Referring to FIG. 1, a functional block diagram is illustrated, of a system 100 for collecting physiological data of a user, such as a user of a respiratory therapy system. The system 100 includes a control system 110, a memory device 114, an electronic interface 119, one or more sensors 130, one or more user devices 170, one or more wearable devices 190, and one or more docking devices 192. In some implementations, the system 100 further optionally includes a respiratory therapy system 120 and/or a blood pressure device 182.

The control system 110 includes one or more processors 112 (hereinafter, processor 112). The control system 110 is generally used to control (e.g., actuate) the various components of the system 100 and/or analyze data obtained and/or generated by the components of the system 100 (e.g., wearable device 190). The processor 112 can be a general or special purpose processor or microprocessor. While one processor 112 is shown in FIG. 1, the control system 110 can include any suitable number of processors (e.g., one processor, two processors, five processors, ten processors, etc.) that can be in a single housing, or located remotely from each other. The control system 110 can be coupled to and/or positioned within, for example, a housing of the user device 170, the wearable device 190, the docking device 192, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of the respiratory device 122, within a housing of the user device 170, within a housing of the wearable device 190, within a housing of the docking device 192, within a housing of one or more of the sensors 130, or any combination thereof. Like the control system 110, the memory device 114 can be centralized (within one such housing) or decentralized (within two or more of such housings, which are physically distinct).

In some implementations, the memory device 114 (FIG. 1) stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more sleep sessions), or any combination thereof. The demographic information can include, for example, information indicative of an age of the user, a gender of the user, a race of the user, an ethnicity of the user, a geographic location of the user, a travel history of the user, a relationship status, a status of whether the user has one or more pets, a status of whether the user has a family, a family history of health conditions, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) test result or score and/or a Pittsburgh Sleep Quality Index (PSQI) score or value. The medical information data can include results from one or more of a polysomnography (PSG) test, a CPAP titration, or a home sleep test (HST), respiratory therapy system settings from one or more sleep sessions, sleep related respiratory events from one or more sleep sessions, or any combination thereof. The self-reported user feedback can include information indicative of a self-reported subjective therapy score (e.g., poor, average, excellent), a self-reported subjective stress level of the user, a self-reported subjective fatigue level of the user, a self-reported subjective health status of the user, a recent life event experienced by the user, or any combination thereof. The user profile information can be updated at any time, such as daily (e.g. between sleep sessions), weekly, monthly or yearly. In some implementations, the memory device 114 stores media content that can be displayed on the display device 128 and/or the display device 172.

The electronic interface 119 is configured to receive data (e.g., physiological data, environmental data, etc.) from the one or more sensors 130 such that the data can be stored in the memory device 114 and/or analyzed by the processor 112 of the control system 110. The received data, such as physiological data, may be used to determine and/or calculate one or more parameters associated with the user, the user's environment, or the like. The electronic interface 119 can communicate with the one or more sensors 130 using a wired connection or a wireless connection (e.g., using an RF communication protocol, a Wi-Fi communication protocol, a Bluetooth communication protocol, an IR communication protocol, over a cellular network, over any other optical communication protocol, etc.). The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. In some implementations, the electronic interface 119 is coupled to or integrated in the user device 170. In other implementations, the electronic interface 119 is coupled to or integrated (e.g., in a housing) with the control system 110, the memory device 114, the wearable device 190, the docking device 192, or any combination thereof.

The respiratory therapy system 120 can include a respiratory pressure therapy (RPT) device 122 (referred to herein as respiratory device 122), a user interface 124, a conduit 126 (also referred to as a tube or an air circuit), a display device 128, a humidification tank 129, a receptacle 180 or any combination thereof. In some implementations, the control system 110, the memory device 114, the display device 128, one or more of the sensors 130, and the humidification tank 129 are part of the respiratory device 122. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 120 is generally used to treat individuals suffering from one or more sleep-related respiratory disorders (e.g., obstructive sleep apnea, central sleep apnea, or mixed sleep apnea).

The respiratory device 122 is generally used to generate pressurized air that is delivered to a user (e.g., using one or more motors that drive one or more compressors). In some implementations, the respiratory device 122 generates continuous constant air pressure that is delivered to the user. In other implementations, the respiratory device 122 generates two or more predetermined pressures (e.g., a first predetermined air pressure and a second predetermined air pressure). In still other implementations, the respiratory device 122 is configured to generate a variety of different air pressures within a predetermined range. For example, the respiratory device 122 can deliver pressurized air at a pressure of at least about 6 cmH₂O, at least about 10 cmH₂O, at least about 20 cmH₂O, between about 6 cmH₂O and about 10 cmH₂O, between about 7 cmH₂O and about 12 cmH₂O, etc. The respiratory device 122 can also deliver pressurized air at a predetermined flow rate between, for example, about −20 L/min and about 150 L/min, while maintaining a positive pressure (relative to the ambient pressure).

The user interface 124 engages a portion of the user's face and delivers pressurized air from the respiratory device 122 to the user's airway to aid in preventing the airway from narrowing and/or collapsing during sleep. This may also increase the user's oxygen intake during sleep. Generally, the user interface 124 engages the user's face such that the pressurized air is delivered to the user's airway via the user's mouth, the user's nose, or both the user's mouth and nose. Together, the respiratory device 122, the user interface 124, and the conduit 126 form an air pathway fluidly coupled with an airway of the user. The pressurized air also increases the user's oxygen intake during sleep.

Depending upon the therapy to be applied, the user interface 124 may form a seal, for example, with a region or portion of the user's face, to facilitate the delivery of gas at a pressure at sufficient variance with ambient pressure to effect therapy, for example, at a positive pressure of about 10 cmH₂O relative to ambient pressure. For other forms of therapy, such as the delivery of oxygen, the user interface may not include a seal sufficient to facilitate delivery to the airways of a supply of gas at a positive pressure of about 10 cmH₂O.

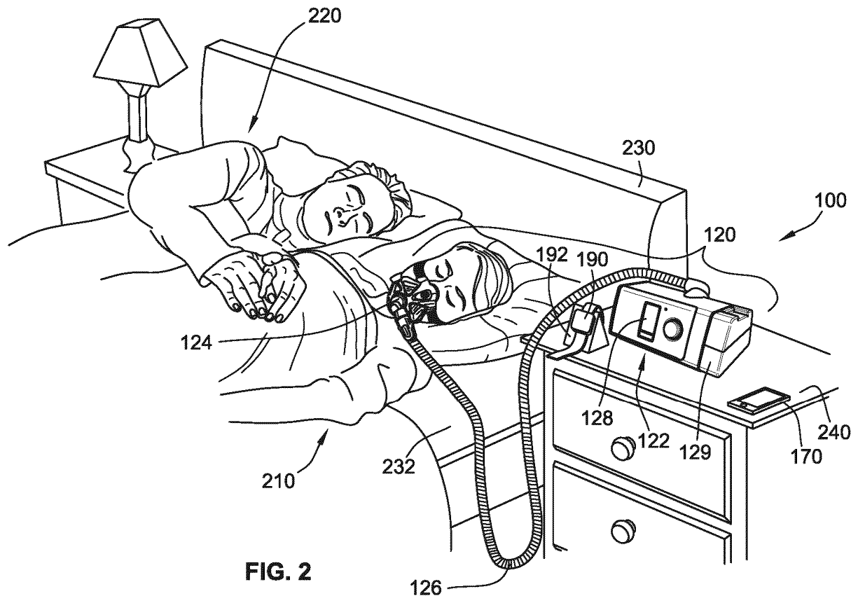

As shown in FIG. 2, in some implementations, the user interface 124 is or includes a facial mask (e.g., a full face mask) that covers the nose and mouth of the user. Alternatively, in some implementations, the user interface 124 is a nasal mask that provides air to the nose of the user or a nasal pillow mask that delivers air directly to the nostrils of the user. The user interface 124 can include a plurality of straps (e.g., including hook and loop fasteners) for positioning and/or stabilizing the interface on a portion of the user (e.g., the face) and a conformal cushion (e.g., silicone, plastic, foam, etc.) that aids in providing an air-tight seal between the user interface 124 and the user. The user interface 124 can also include one or more vents for permitting the escape of carbon dioxide and other gases exhaled by the user 210. In other implementations, the user interface 124 includes a mouthpiece (e.g., a night guard mouthpiece molded to conform to the user's teeth, a mandibular repositioning device, etc.).

The conduit 126 (also referred to as an air circuit or tube) allows the flow of air between two components of the respiratory therapy system 120, such as the respiratory device 122 and the user interface 124. In some implementations, there can be separate limbs of the conduit for inhalation and exhalation. In other implementations, a single limb conduit is used for both inhalation and exhalation.

One or more of the respiratory device 122, the user interface 124, the conduit 126, the display device 128, and the humidification tank 129 can contain one or more sensors (e.g., a pressure sensor, a flow rate sensor, a humidity sensor, a temperature sensor, or more generally any of the other sensors 130 described herein). These one or more sensors can be used, for example, to measure the air pressure and/or flow rate of pressurized air supplied by the respiratory device 122.

The display device 128 is generally used to display image(s) including still images, video images, or both and/or information regarding the respiratory device 122. For example, the display device 128 can provide information regarding the status of the respiratory device 122 (e.g., whether the respiratory device 122 is on/off, the pressure of the air being delivered by the respiratory device 122, the temperature of the air being delivered by the respiratory device 122, etc.) and/or other information (e.g., a sleep score and/or a therapy score (such as a myAir™ score, such as described in WO 2016/061629, which is hereby incorporated by reference herein in its entirety), the current date/time, personal information for the user 210, etc.). In some implementations, the display device 128 acts as a human-machine interface (HMI) that includes a graphic user interface (GUI) configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory device 122.

The humidification tank 129 is coupled to or integrated in the respiratory device 122. The humidification tank 129 includes a reservoir of water that can be used to humidify the pressurized air delivered from the respiratory device 122. The respiratory device 122 can include a heater to heat the water in the humidification tank 129 in order to humidify the pressurized air provided to the user. Additionally, in some implementations, the conduit 126 can also include a heating element (e.g., coupled to and/or imbedded in the conduit 126) that heats the pressurized air delivered to the user. The humidification tank 129 can be fluidly coupled to a water vapor inlet of the air pathway and deliver water vapor into the air pathway via the water vapor inlet, or can be formed in-line with the air pathway as part of the air pathway itself. In other implementations, the respiratory device 122 or the conduit 126 can include a waterless humidifier. The waterless humidifier can incorporate sensors that interface with other sensor positioned elsewhere in system 100.

In some implementations, the system 100 can be used to deliver at least a portion of a substance from a receptacle 180 to the air pathway the user based at least in part on the physiological data, the sleep-related parameters, other data or information, or any combination thereof. Generally, modifying the delivery of the portion of the substance into the air pathway can include (i) initiating the delivery of the substance into the air pathway, (ii) ending the delivery of the portion of the substance into the air pathway, (iii) modifying an amount of the substance delivered into the air pathway, (iv) modifying a temporal characteristic of the delivery of the portion of the substance into the air pathway, (v) modifying a quantitative characteristic of the delivery of the portion of the substance into the air pathway, (vi) modifying any parameter associated with the delivery of the substance into the air pathway, or (vii) any combination of (i)-(vi).

Modifying the temporal characteristic of the delivery of the portion of the substance into the air pathway can include changing the rate at which the substance is delivered, starting and/or finishing at different times, continuing for different time periods, changing the time distribution or characteristics of the delivery, changing the amount distribution independently of the time distribution, etc. The independent time and amount variation ensures that, apart from varying the frequency of the release of the substance, one can vary the amount of substance released each time. In this manner, a number of different combination of release frequencies and release amounts (e.g., higher frequency but lower release amount, higher frequency and higher amount, lower frequency and higher amount, lower frequency and lower amount, etc.) can be achieved. Other modifications to the delivery of the portion of the substance into the air pathway can also be utilized.

The respiratory therapy system 120 can be used, for example, as a ventilator or a positive airway pressure (PAP) system such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

Referring to FIG. 2, a portion of the system 100 (FIG. 1), according to some implementations, is illustrated. A user 210 of the respiratory therapy system 120 and a bed partner 220 are located in a bed 230 and are laying on a mattress 232. A sensor (e.g., any number of one or more sensors 130) can be used to generate or monitor various parameters during a respiratory therapy, sleep therapy, sleeping, and/or resting session of the user 210, such as sensor(s) incorporated in the user device 170, the wearable device 190, the docking device 192, the respiratory device 122, or any combination thereof. Certain aspects of the present disclosure can relate to facilitating data collection for any individual, such as an individual using a respiratory therapy device (e.g., user 210) or an individual not using a respiratory therapy device (e.g., bed partner 220).

The user interface 124 is a facial mask (e.g., a full face mask) that covers the nose and mouth of the user 210. Alternatively, the user interface 124 can be a nasal mask that provides air to the nose of the user 210 or a nasal pillow mask that delivers air directly to the nostrils of the user 210. The user interface 124 can include a plurality of straps (e.g., including hook and loop fasteners) for positioning and/or stabilizing the interface on a portion of the user 210 (e.g., the face) and a conformal cushion (e.g., silicone, plastic, foam, etc.) that aids in providing an air-tight seal between the user interface 124 and the user 210. The user interface 124 can also include one or more vents for permitting the escape of carbon dioxide and other gases exhaled by the user 210. In other implementations, the user interface 124 is or includes a mouthpiece (e.g., a night guard mouthpiece molded to conform to the user's teeth, a mandibular repositioning device, etc.).

The user interface 124 is fluidly coupled and/or connected to the respiratory device 122 via the conduit 126. In turn, the respiratory device 122 delivers pressurized air to the user 210 via the conduit 126 and the user interface 124 to increase the air pressure in the throat of the user 210 to aid in preventing the airway from closing and/or narrowing during sleep. The respiratory device 122 can be positioned on a nightstand 240 that is directly adjacent to the bed 230 as shown in FIG. 2, or more generally, on any surface or structure that is generally adjacent to the bed 230 and/or the user 210.

Generally, a user who is prescribed usage of the respiratory therapy system 120 will tend to experience higher quality sleep and less fatigue during the day after using the respiratory therapy system 120 during the sleep compared to not using the respiratory therapy system 120 (especially when the user suffers from sleep apnea or other sleep related disorders). For example, the user 210 may suffer from obstructive sleep apnea and rely on the user interface 124 (e.g., a full face mask) to deliver pressurized air from the respiratory device 122 via conduit 126. The respiratory device 122 can be a continuous positive airway pressure (CPAP) machine used to increase air pressure in the throat of the user 210 to prevent the airway from closing and/or narrowing during sleep. For someone with sleep apnea, their airway can narrow or collapse during sleep, reducing oxygen intake, and forcing them to wake up and/or otherwise disrupt their sleep. The CPAP machine prevents the airway from narrowing or collapsing, thus minimizing the occurrences where she wakes up or is otherwise disturbed due to reduction in oxygen intake. While the respiratory device 122 strives to maintain a medically prescribed air pressure or pressures during sleep, the user can experience sleep discomfort due to the therapy.

Referring to back to FIG. 1, the one or more sensors 130 of the system 100 include a pressure sensor 132, a flow rate sensor 134, temperature sensor 136, a motion sensor 138, a microphone 140, a speaker 142, a radio-frequency (RF) receiver 146, a RF transmitter 148, a camera 150, an infrared sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, an analyte sensor 174, a moisture sensor 176, a Light Detection and Ranging (LiDAR) sensor 178, an electrodermal sensor, an accelerometer, an electrooculography (EOG) sensor, a light sensor, a humidity sensor, an air quality sensor, or any combination thereof. Generally, each of the one or more sensors 130 are configured to output sensor data that is received and stored in the memory device 114 or one or more other memory devices.

While the one or more sensors 130 are shown and described as including each of the pressure sensor 132, the flow rate sensor 134, the temperature sensor 136, the motion sensor 138, the microphone 140, the speaker 142, the RF receiver 146, the RF transmitter 148, the camera 150, the infrared sensor 152, the photoplethysmogram (PPG) sensor 154, the electrocardiogram (ECG) sensor 156, the electroencephalography (EEG) sensor 158, the capacitive sensor 160, the force sensor 162, the strain gauge sensor 164, the electromyography (EMG) sensor 166, the oxygen sensor 168, the analyte sensor 174, the moisture sensor 176, and the Light Detection and Ranging (LiDAR) sensor 178 more generally, the one or more sensors 130 can include any combination and any number of each of the sensors described and/or shown herein.

Data from room environment sensors can also be used, such as to extract environmental parameters from sensor data. Example environmental parameters can include temperature before and/or throughout a sleep session (e.g., too warm, too cold), humidity (e.g., too high, too low), pollution levels (e.g., an amount and/or concentration of CO₂ and/or particulates being under or over a threshold), light levels (e.g., too bright, not using blackout blinds, too much blue light before falling asleep), sound levels (e.g., above a threshold, types of sources, linked to interruptions in sleep, snoring of a partner), and air quality (e.g., types of particulates in a room that may cause allergies or other effects, such as pollution from pets, dust mites, and others). These parameters can be obtained via sensors on a respiratory device 122, via sensors on a user device 170 (e.g., connected via Bluetooth or internet), via sensors on a wearable device 190, via sensors on a docking device 192, via separate sensors (such as connected to a home automation system), or any combination thereof. Such environmental data can be used to improve analysis of non-environmental data (e.g., physiological data) and/or to otherwise facilitate changing modes of a wearable device 190. For example, a wearable device 190 can leverage environmental data to confirm that it is located in a specific location (e.g., a bedroom) designated for docking with the docking device 192.

As described herein, the system 100 generally can be used to generate data (e.g., physiological data, environmental data, etc.) associated with a user (e.g., a user of the respiratory therapy system 120 shown in FIG. 2 or any other suitable user) before, during, and/or after a sleep session. The generated data can be analyzed to extract one or more parameters, including physiological parameters (e.g., heart rate, heart rate variability, temperature, temperature variability, respiration rate, respiration rate variability, breath morphology, EEG activity, EMG activity, ECG data, and the like), environmental parameters associated with the user's environment (e.g., a sleep environment), and the like. Physiological parameters can include sleep-related parameters associated with a sleep session as well as non-sleep related parameters. Examples of one or more sleep-related parameters that can be determined for a user during the sleep session include an Apnea-Hypopnea Index (AHI) score, a sleep score, a therapy score, a flow signal, a pressure signal, a respiration signal, a respiration rate, an inspiration amplitude, an expiration amplitude, an inspiration-expiration ratio, a number of events (e.g. apnea events) per hour, a pattern of events, a sleep state and/or sleep stage, a heart rate, a heart rate variability, movement of the user 210, temperature, EEG activity, EMG activity, arousal, snoring, choking, coughing, whistling, wheezing, or any combination thereof.

The one or more sensors 130 can be used to generate, for example, physiological data, environmental data, flow rate data, pressure data, motion data, acoustic data, etc. In some implementations, the data generated by one or more of the sensors 130 can be used by the control system 110 to determine the duration of sleep and sleep quality of user 210. For example, a sleep-wake signal associated with the user 210 during the sleep session and one or more sleep-related parameters. The sleep-wake signal can be indicative of one or more sleep states, including sleep, wakefulness, relaxed wakefulness, micro-awakenings, or distinct sleep stages such as a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more of the sensors, such as sensors 130, are described in, for example, WO 2014/047310, US 2014/0088373, WO 2017/132726, WO 2019/122413, and WO 2019/122414, each of which is hereby incorporated by reference herein in its entirety.

The sleep-wake signal can also be timestamped to determine a time that the user enters the bed, a time that the user exits the bed, a time that the user attempts to fall asleep, etc. The sleep-wake signal can be measured by the one or more sensors 130 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory device 122, or any combination thereof during the sleep session.

The event(s) can include snoring, apneas (e.g., central apneas, obstructive apneas, mixed apneas, and hypopneas), a mouth leak, a mask leak (e.g., from the user interface 124), a restless leg, a sleeping disorder, choking, an increased heart rate, a heart rate variation, labored breathing, an asthma attack, an epileptic episode, a seizure, a fever, a cough, a sneeze, a snore, a gasp, the presence of an illness such as the common cold or the flu, or any combination thereof. In some implementations, mouth leak can include continuous mouth leak, or valve-like mouth leak (i.e. varying over the breath duration) where the lips of a user, typically using a nasal/nasal pillows mask, pop open on expiration. Mouth leak can lead to dryness of the mouth, bad breath, and is sometimes colloquially referred to as “sandpaper mouth.”

The one or more sleep-related parameters that can be determined for the user during the sleep session based on the sleep-wake signal include, for example, sleep quality metrics such as a total time in bed, a total sleep time, a sleep onset latency, a wake-after-sleep-onset parameter, a sleep efficiency, a fragmentation index, or any combination thereof.

The data generated by the one or more sensors 130 (e.g., physiological data, environmental data, flow rate data, pressure data, motion data, acoustic data, etc.) can also be used to determine a respiration signal. The respiration signal is generally indicative of respiration or breathing of the user. The respiration signal can be indicative of, for example, a respiration rate, a respiration rate variability, an inspiration amplitude, an expiration amplitude, an inspiration-expiration ratio, and other respiration-related parameters, as well as any combination thereof. In some cases, during a sleep session, the respiration signal can include a number of events per hour (e.g., during sleep), a pattern of events, pressure settings of the respiratory device 122, or any combination thereof. The event(s) can include snoring, apneas, central apneas, obstructive apneas, mixed apneas, hypopneas, a mouth leak, a mask leak (e.g., from the user interface 124), a restless leg, a sleeping disorder, choking, an increased heart rate, labored breathing, an asthma attack, an epileptic episode, a seizure, or any combination thereof.

Generally, the sleep session includes any point in time after the user 210 has laid or sat down in the bed 230 (or another area or object on which they intend to sleep), and/or has turned on the respiratory device 122 and/or donned the user interface 124. The sleep session can thus include time periods (i) when the user 210 is using the CPAP system but before the user 210 attempts to fall asleep (for example when the user 210 lays in the bed 230 reading a book); (ii) when the user 210 begins trying to fall asleep but is still awake; (iii) when the user 210 is in a light sleep (also referred to as stage 1 and stage 2 of non-rapid eye movement (NREM) sleep); (iv) when the user 210 is in a deep sleep (also referred to as slow-wave sleep, SWS, or stage 3 of NREM sleep); (v) when the user 210 is in rapid eye movement (REM) sleep; (vi) when the user 210 is periodically awake between light sleep, deep sleep, or REM sleep; or (vii) when the user 210 wakes up and does not fall back asleep.

The sleep session is generally defined as ending once the user 210 removes the user interface 124, turns off the respiratory device 122, and/or gets out of bed 230. In some implementations, the sleep session can include additional periods of time, or can be limited to only some of the above-disclosed time periods. For example, the sleep session can be defined to encompass a period of time beginning when the respiratory device 122 begins supplying the pressurized air to the airway or the user 210, ending when the respiratory device 122 stops supplying the pressurized air to the airway of the user 210, and including some or all of the time points in between, when the user 210 is asleep or awake.

The pressure sensor 132 outputs pressure data that can be stored in the memory device 114 and/or analyzed by the processor 112 of the control system 110. In some implementations, the pressure sensor 132 is an air pressure sensor (e.g., barometric pressure sensor) that generates sensor data indicative of the respiration (e.g., inhaling and/or exhaling) of the user of the respiratory therapy system 120 and/or ambient pressure. In such implementations, the pressure sensor 132 can be coupled to or integrated in the respiratory device 122. the user interface 124, or the conduit 126. The pressure sensor 132 can be used to determine an air pressure in the respiratory device 122, an air pressure in the conduit 126, an air pressure in the user interface 124, or any combination thereof. The pressure sensor 132 can be, for example, a capacitive sensor, an electromagnetic sensor, an inductive sensor, a resistive sensor, a piezoelectric sensor, a strain-gauge sensor, an optical sensor, a potentiometric sensor, or any combination thereof. In one example, the pressure sensor 132 can be used to determine a blood pressure of a user.

The flow rate sensor 134 outputs flow rate data that can be stored in the memory device 114 and/or analyzed by the processor 112 of the control system 110. In some implementations, the flow rate sensor 134 is used to determine an air flow rate from the respiratory device 122, an air flow rate through the conduit 126, an air flow rate through the user interface 124, or any combination thereof. In such implementations, the flow rate sensor 134 can be coupled to or integrated in the respiratory device 122, the user interface 124, or the conduit 126. The flow rate sensor 134 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof.

The flow rate sensor 134 can be used to generate flow rate data associated with the user 210 (FIG. 2) of the respiratory device 122 during the sleep session. Examples of flow rate sensors (such as, for example, the flow rate sensor 134) are described in WO 2012/012835, which is hereby incorporated by reference herein in its entirety. In some implementations, the flow rate sensor 134 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user.

The temperature sensor 136 outputs temperature data that can be stored in the memory device 114 and/or analyzed by the processor 112 of the control system 110. In some implementations, the temperature sensor 136 generates temperature data indicative of a core body temperature of the user 210 (FIG. 2), a skin temperature of the user 210, a temperature of the air flowing from the respiratory device 122 and/or through the conduit 126, a temperature of the air in the user interface 124, an ambient temperature, or any combination thereof. The temperature sensor 136 can be, for example, a thermocouple sensor, a thermistor sensor, a silicon band gap temperature sensor or semiconductor-based sensor, a resistance temperature detector, or any combination thereof.

The motion sensor 138 outputs motion data that can be stored in the memory device 114 and/or analyzed by the processor 112 of the control system 110. The motion sensor 138 can be used to detect movement of the user 210 during the sleep session, and/or detect movement of any of the components of the respiratory therapy system 120, such as the respiratory device 122, the user interface 124, or the conduit 126. The motion sensor 138 can include one or more inertial sensors, such as accelerometers, gyroscopes, and magnetometers. In some implementations, the motion sensor 138 alternatively or additionally generates one or more signals representing bodily movement of the user, from which may be obtained a signal representing a sleep state or sleep stage of the user; for example, via a respiratory movement of the user. In some implementations, the motion data from the motion sensor 138 can be used in conjunction with additional data from another sensor 130 to determine the sleep state or sleep stage of the user. In some implementations, the motion data can be used to determine a location, a body position, and/or a change in body position of the user. In some cases, a motion sensor 138 incorporated in a wearable device 190 may be automatically used when the wearable device 190 is worn by the user 210, but may be automatically not used when the wearable device 190 is docked with the docking device 192, in which case one or more other sensors may optionally be used instead.

The microphone 140 outputs sound data that can be stored in the memory device 114 and/or analyzed by the processor 112 of the control system 110. The microphone 140 can be used to record sound(s) during a sleep session (e.g., sounds from the user 210) to determine (e.g., using the control system 110) one or more sleep related parameters, which may include one or more events (e.g., respiratory events), as described in further detail herein. The microphone 140 can be coupled to or integrated in the respiratory device 122, the user interface 124, the conduit 126, the user device 170, the wearable device 190, or the docking device 192. In some implementations, the system 100 includes a plurality of microphones (e.g., two or more microphones and/or an array of microphones with beamforming) such that sound data generated by each of the plurality of microphones can be used to discriminate the sound data generated by another of the plurality of microphones. In an example, when operating in a first mode (e.g., worn mode), the wearable device 190 may collect data via an onboard microphone, however when operating in a second mode (e.g., a docked mode), the wearable device 190 may cease collecting data via the onboard microphone and instead collect similar data via a microphone incorporated in the docking device 192.

The speaker 142 outputs sound waves. In one or more implementations, the sound waves can be audible to a user of the system 100 (e.g., the user 210 of FIG. 2) or inaudible to the user of the system (e.g., ultrasonic sound waves). The speaker 142 can be used, for example, as an alarm clock or to play an alert or message to the user 210 (e.g., in response to an identified body position and/or a change in body position). In some implementations, the speaker 142 can be used to communicate the audio data generated by the microphone 140 to the user. The speaker 142 can be coupled to or integrated in the respiratory device 122, the user interface 124, the conduit 126, the user device 170, the wearable device 190, or the docking device 192. In an example, when operating in a first mode (e.g., worn mode), the wearable device 190 may output signals via an onboard speaker, however when operating in a second mode (e.g., a docked mode), the wearable device 190 may cease outputting signals via the onboard speaker and instead output similar signals via a speaker incorporated in the docking device 192.

The microphone 140 and the speaker 142 can be used as separate devices. In some implementations, the microphone 140 and the speaker 142 can be combined into an acoustic sensor 141 (e.g. a SONAR sensor), as described in, for example, WO 2018/050913 and WO 2020/104465, each of which is hereby incorporated by reference herein in its entirety. In such implementations, the speaker 142 generates or emits sound waves at a predetermined interval and/or frequency and the microphone 140 detects the reflections of the emitted sound waves from the speaker 142. In one or more implementations, the sound waves generated or emitted by the speaker 142 can have a frequency that is not audible to the human ear (e.g., below 20 Hz or above around 18 kHz) so as not to disturb the sleep of the user 210 or the bed partner 220 (FIG. 2). Based at least in part on the data from the microphone 140 and/or the speaker 142, the control system 110 can determine a location of the user 210 (FIG. 2) and/or one or more of the sleep-related parameters (including e.g., an identified body position and/or a change in body position) and/or respiration-related parameters described in herein such as, for example, a respiration signal (from which e.g., breath morphology may be determined), a respiration rate, an inspiration amplitude, an expiration amplitude, an inspiration-expiration ratio, a number of events per hour, a pattern of events, a sleep state, a sleep stage, or any combination thereof. In this context, a sonar sensor may be understood to concern an active acoustic sensing, such as by generating/transmitting ultrasound or low frequency ultrasound sensing signals (e.g., in a frequency range of about 17-23 kHz, 18-22 kHz, or 17-18 kHz, for example), through the air. Such a system may be considered in relation to WO2018/050913 and WO 2020/104465 mentioned above.

In some implementations, the sensors 130 include (i) a first microphone that is the same as, or similar to, the microphone 140, and is integrated in the acoustic sensor 141 and (ii) a second microphone that is the same as, or similar to, the microphone 140, but is separate and distinct from the first microphone that is integrated in the acoustic sensor 141.

The RF transmitter 148 generates and/or emits radio waves having a predetermined frequency and/or a predetermined amplitude (e.g., within a high frequency band, within a low frequency band, long wave signals, short wave signals, etc.). The RF receiver 146 detects the reflections of the radio waves emitted from the RF transmitter 148, and this data can be analyzed by the control system 110 to determine a location and/or a body position of the user 210 (FIG. 2) and/or one or more of the sleep-related parameters described herein. An RF receiver (either the RF receiver 146 and the RF transmitter 148 or another RF pair) can also be used for wireless communication between the control system 110, the respiratory device 122, the one or more sensors 130, the user device 170, the wearable device 190, the docking device 192, or any combination thereof. While the RF receiver 146 and RF transmitter 148 are shown as being separate and distinct elements in FIG. 1, in some implementations, the RF receiver 146 and RF transmitter 148 are combined as a part of an RF sensor 147 (e.g. a RADAR sensor). In some such implementations, the RF sensor 147 includes a control circuit. The specific format of the RF communication could be Wi-Fi, Bluetooth, or etc.

In some implementations, the RF sensor 147 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that the is the same as, or similar to, the RF sensor 147. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 150 outputs image data reproducible as one or more images (e.g., still images, video images, thermal images, or any combination thereof) that can be stored in the memory device 114. The image data from the camera 150 can be used by the control system 110 to determine one or more of the sleep-related parameters described herein. The image data from the camera 150 can be used by the control system 110 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 150 can be used to identify a location and/or a body position of the user, to determine chest movement of the user 210, to determine air flow of the mouth and/or nose of the user 210, to determine a time when the user 210 enters the bed 230, and to determine a time when the user 210 exits the bed 230. The camera 150 can also be used to track eye movements, pupil dilation (if one or both of the user 210's eyes are open), blink rate, or any changes during REM sleep.

The infrared (IR) sensor 152 outputs infrared image data reproducible as one or more infrared images (e.g., still images, video images, or both) that can be stored in the memory device 114. The infrared data from the IR sensor 152 can be used to determine one or more sleep-related parameters during a sleep session, including a temperature of the user 210 and/or movement of the user 210. The IR sensor 152 can also be used in conjunction with the camera 150 when measuring the presence, location, and/or movement of the user 210. The IR sensor 152 can detect infrared light having a wavelength between about 700 nm and about 1 mm, for example, while the camera 150 can detect visible light having a wavelength between about 380 nm and about 740 nm.

The PPG sensor 154 outputs physiological data associated with the user 210 (FIG. 2) that can be used to determine one or more sleep-related parameters, such as, for example, a heart rate, a heart rate pattern, a heart rate variability, a cardiac cycle, respiration rate, an inspiration amplitude, an expiration amplitude, an inspiration-expiration ratio, estimated blood pressure parameter(s), or any combination thereof. The PPG sensor 154 can be worn by the user 210 (e.g., incorporated in a wearable device 190), embedded in clothing and/or fabric that is worn by the user 210, embedded in and/or coupled to the user interface 124 and/or its associated headgear (e.g., straps, etc.), etc.

The ECG sensor 156 outputs physiological data associated with electrical activity of the heart of the user 210. In some implementations, the ECG sensor 156 includes one or more electrodes that are positioned on or around a portion of the user 210 during the sleep session. The physiological data from the ECG sensor 156 can be used, for example, to determine one or more of the sleep-related parameters described herein.

The EEG sensor 158 outputs physiological data associated with electrical activity of the brain of the user 210. In some implementations, the EEG sensor 158 includes one or more electrodes that are positioned on or around the scalp of the user 210 during the sleep session. The physiological data from the EEG sensor 158 can be used, for example, to determine a sleep state or sleep stage of the user 210 at any given time during the sleep session. In some implementations, the EEG sensor 158 can be integrated in the user interface 124, the associated headgear (e.g., straps, etc.), a wearable device 190, or the like.

The capacitive sensor 160, the force sensor 162, and the strain gauge sensor 164 output data that can be stored in the memory device 114 and used by the control system 110 to determine one or more of the sleep-related parameters described herein. The EMG sensor 166 outputs physiological data associated with electrical activity produced by one or more muscles. The oxygen sensor 168 outputs oxygen data indicative of an oxygen concentration of gas (e.g., in the conduit 126 or at the user interface 124). The oxygen sensor 168 can be, for example, an ultrasonic oxygen sensor, an electrical oxygen sensor, a chemical oxygen sensor, an optical oxygen sensor, or any combination thereof.

The analyte sensor 174 can be used to detect the presence of an analyte in the exhaled breath of the user 210. The data output by the analyte sensor 174 can be stored in the memory device 114 and used by the control system 110 to determine the identity and concentration of any analytes in the user 210's breath. In some implementations, the analyte sensor 174 is positioned near the user 210's mouth to detect analytes in breath exhaled from the user 210's mouth. For example, when the user interface 124 is a facial mask that covers the nose and mouth of the user 210, the analyte sensor 174 can be positioned within the facial mask to monitor the user 210's mouth breathing. In other implementations, such as when the user interface 124 is a nasal mask or a nasal pillow mask, the analyte sensor 174 can be positioned near the user 210's nose to detect analytes in breath exhaled through the user's nose. In still other implementations, the analyte sensor 174 can be positioned near the user 210's mouth when the user interface 124 is a nasal mask or a nasal pillow mask. In some implementations, the analyte sensor 174 can be used to detect whether any air is inadvertently leaking from the user 210's mouth. In some implementations, the analyte sensor 174 is a volatile organic compound (VOC) sensor that can be used to detect carbon-based chemicals or compounds. In some implementations, the analyte sensor 174 can also be used to detect whether the user 210 is breathing through their nose or mouth. For example, if the data output by an analyte sensor 174 positioned near the user 210's mouth or within the facial mask (in implementations where the user interface 124 is a facial mask) detects the presence of an analyte, the control system 110 can use this data as an indication that the user 210 is breathing through their mouth.

The moisture sensor 176 outputs data that can be stored in the memory device 114 and used by the control system 110. The moisture sensor 176 can be used to detect moisture in various areas surrounding the user (e.g., inside the conduit 126 or the user interface 124, near the user 210's face, near the connection between the conduit 126 and the user interface 124, near the connection between the conduit 126 and the respiratory device 122, etc.). Thus, in some implementations, the moisture sensor 176 can be positioned in the user interface 124 or in the conduit 126 to monitor the humidity of the pressurized air from the respiratory device 122. In other implementations, the moisture sensor 176 is placed near any area where moisture levels need to be monitored. The moisture sensor 176 can also be used to monitor the humidity of the ambient environment surrounding the user 210, for example, the air inside the user 210's bedroom. The moisture sensor 176 can also be used to track the user 210's biometric response to environmental changes.

One or more Light Detection and Ranging (LiDAR) sensors 178 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 178 can measure and map an area extending 5 meters or more away from the sensor. The LiDAR data can be fused with point cloud data estimated by an electromagnetic RADAR sensor, for example. The LiDAR sensor(s) 178 may also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 130 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, an orientation sensor, a rain sensor, a soil moisture sensor, a water flow sensor, an alcohol sensor, or any combination thereof.

While shown separately in FIG. 1, any combination of the one or more sensors 130 can be integrated in and/or coupled to any one or more of the components of the system 100, including the respiratory device 122, the user interface 124, the conduit 126, the humidification tank 129, the control system 110, the user device 170, the wearable device 190, the docking device 192, or any combination thereof. For example, one or more acoustic sensors 141 can be integrated in and/or coupled to both the wearable device 190 and the docking device 192. In such implementations, the wearable device 190 may collect acoustic data while being worn, but upon docking the wearable device 190 with the docking device 192, the docking device 192 may take over collection of the acoustic data using its own acoustic sensor(s) 141. In some implementations, at least one of the one or more sensors 130 is not physically and/or communicatively coupled to the respiratory device 122, the control system 110, the user device 170, the wearable device 190, or the docking device 192, and is positioned generally adjacent to the user 210 during the sleep session (e.g., positioned on or in contact with a portion of the user 210, worn by the user 210, coupled to or positioned on the nightstand, coupled to the mattress, coupled to the ceiling, etc.).

The data from the one or more sensors 130 can be analyzed to determine one or more parameters, such as physiological parameters, environmental parameters, and the like, as disclosed in further detail herein. In some cases, one or more physiological parameters can include a respiration signal, a respiration rate, a respiration pattern or morphology, respiration rate variability, an inspiration amplitude, an expiration amplitude, an inspiration-expiration ratio, a length of time between breaths, a time of maximal inspiration, a time of maximal expiration, a forced breath parameter (e.g., distinguishing releasing breath from forced exhalation), an occurrence of one or more events, a number of events per hour, a pattern of events, a sleep state, a sleep stage, an apnea-hypopnea index (AHI), a heart rate, heart rate variability, movement of the user 210, temperature, EEG activity, EMG activity, ECG data, a sympathetic response parameter, a parasympathetic response parameter or any combination thereof. The one or more events can include snoring, apneas, central apneas, obstructive apneas, mixed apneas, hypopneas, an intentional mask leak, an unintentional mask leak, a mouth leak, a cough, a restless leg, a sleeping disorder, choking, an increased heart rate, labored breathing, an asthma attack, an epileptic episode, a seizure, increased blood pressure, or any combination thereof. Many of these physiological parameters are sleep-related parameters, although in some cases the data from the one or more sensors 130 can be analyzed to determine one or more non-physiological parameters, such as non-physiological sleep-related parameters. Non-physiological parameters can include environmental parameters. Non-physiological parameters can also include operational parameters of the respiratory therapy system, including flow rate, pressure, humidity of the pressurized air, speed of motor, etc. Other types of physiological and non-physiological parameters can also be determined, either from the data from the one or more sensors 130, or from other types of data.

The user device 170 (FIG. 1) includes a display device 172. The user device 170 can be, for example, a mobile device such as a smart phone, a tablet, a gaming console, a smart watch, a laptop, or the like. Alternatively, the user device 170 can be an external sensing system, a television (e.g., a smart television) or another smart home device (e.g., a smart speaker(s), optionally with a display, such as Google Home™, Google Nest™, Amazon Echo™, Amazon Echo Show™, Alexa™-enabled devices, etc.). In some implementations, the user device is a wearable device (e.g., a smart watch), such as wearable device 190. The display device 172 is generally used to display image(s) including still images, video images, or both. In some implementations, the display device 172 acts as a human-machine interface (HMI) that includes a graphic user interface (GUI) configured to display the image(s) and an input interface. The display device 172 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 170. In some implementations, one or more user devices can be used by and/or included in the system 100.

The blood pressure device 182 is generally used to aid in generating physiological data for determining one or more blood pressure measurements associated with a user. The blood pressure device 182 can include at least one of the one or more sensors 130 to measure, for example, a systolic blood pressure component and/or a diastolic blood pressure component. In some cases, the blood pressure device 182 is a wearable device, such as wearable device 190.

In some implementations, the blood pressure device 182 is a sphygmomanometer including an inflatable cuff that can be worn by a user and a pressure sensor (e.g., the pressure sensor 132 described herein). For example, the blood pressure device 182 can be worn on an upper arm of the user. In such implementations where the blood pressure device 182 is a sphygmomanometer, the blood pressure device 182 also includes a pump (e.g., a manually operated bulb) for inflating the cuff. In some implementations, the blood pressure device 182 is coupled to the respiratory device 122 of the respiratory therapy system 120, which in turn delivers pressurized air to inflate the cuff More generally, the blood pressure device 182 can be communicatively coupled with, and/or physically integrated in (e.g., within a housing), the control system 110, the memory device 114, the respiratory therapy system 120, the user device 170, the wearable device 190 and/or the docking device 192.

The wearable device 190 is generally used to aid in generating physiological data associated with the user by collecting information from the user (e.g., by sensing blood oxygenation using a PPG sensor 154) or by otherwise tracking information associated with movement or environment of the user. Examples of data acquired by the wearable device 190 includes, for example, a number of steps, a distance traveled, a number of steps climbed, a duration of physical activity, a type of physical activity, an intensity of physical activity, time spent standing, a respiration rate, an average respiration rate, a resting respiration rate, a maximum respiration rate, a respiration rate variability, a heart rate, an average heart rate, a resting heart rate, a maximum heart rate, a heart rate variability, a number of calories burned, blood oxygen saturation level (SpO₂), electrodermal activity (also known as skin conductance or galvanic skin response), a position of the user, a posture of the user, or any combination thereof. The wearable device 190 includes one or more of the sensors 130 described herein, such as, for example, the motion sensor 138 (e.g., one or more accelerometers and/or gyroscopes), the PPG sensor 154, and/or the ECG sensor 156.

In some implementations, the wearable device 190 can be worn by the user, such as a smartwatch, a wristband, a ring, or a patch. For example, referring to FIG. 2, the wearable device 190 is a smartwatch capable of being worn on a wrist of the user 210 or, as depicted in FIG. 2, docked on a docking device 192 when not worn. The wearable device 190 can also be coupled to or integrated into a garment or clothing that is worn by the user. Alternatively still, the wearable device 190 can also be coupled to or integrated in (e.g., within the same housing) the user device 170. More generally, the wearable device 190 can be communicatively coupled with, or physically integrated in (e.g., within a housing), the control system 110, the memory device 114, the respiratory therapy system 120, the user device 170, the docking device 192, and/or the blood pressure device 182.

While the control system 110 and the memory device 114 are described and shown in FIG. 1 as being a separate and distinct component of the system 100, in some implementations, the control system 110 and/or the memory device 114 are integrated in the user device 170, the respiratory device 122, the wearable device 190, and/or the docking device 192. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc.), or any combination thereof.

While system 100 is shown as including all of the components described above, more or fewer components can be included in a system for collecting data associated with a user, according to implementations of the present disclosure. For example, a first alternative system includes the control system 110, the memory device 114, the wearable device 190, the docking device 192, and at least one of the one or more sensors 130. As another example, a second alternative system includes the control system 110, the memory device 114, the wearable device 190, the docking device 192, at least one of the one or more sensors 130, the user device 170, and the blood pressure device 182. As yet another example, a third alternative system includes the control system 110, the memory device 114, the respiratory therapy system 120, the wearable device 190, the docking device 192, at least one of the one or more sensors 130, and the user device 170. As a further example, a fourth alternative system includes the control system 110, the memory device 114, the respiratory therapy system 120, at least one of the one or more sensors 130, the user device 170, the wearable device 190, and the docking device 192. Thus, various systems can be formed using any portion or portions of the components shown and described herein and/or in combination with one or more other components.

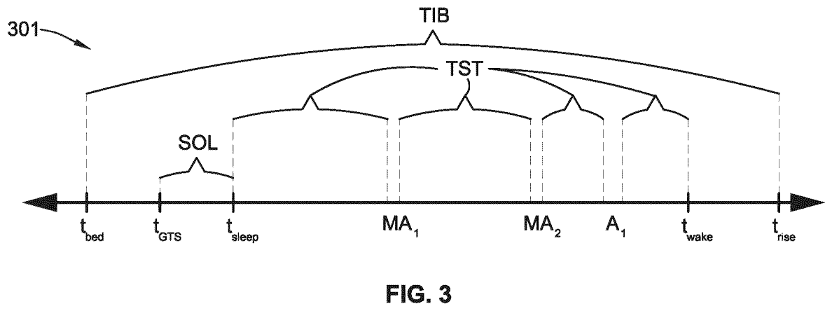

Referring to the timeline 301 in FIG. 3, the enter bed time t_{bed} is associated with the time that the user initially enters the bed (e.g., bed 230 in FIG. 2) prior to falling asleep (e.g., when the user lies down or sits in the bed). The enter bed time t_{bed} can be identified based on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (GTS) is associated with the time that the user initially attempts to fall asleep after entering the bed (t_{bed}). For example, after entering the bed, the user may engage in one or more activities to wind down prior to trying to sleep (e.g., reading, watching TV, listening to music, using the user device 170, etc.). In some cases, one or both of t_{bed} can be based at least in part on detection of a docking event between a wearable device and a docking device (e.g., indicating in some cases that the user is taking off the wearable device for the night and charging it next to the user's bed). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 4 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based on a rise threshold duration (e.g., the user has left the bed for at least 15 minutes, at least 20 minutes, at least 30 minutes, at least 1 hour, etc.). In some cases, tᵣᵢₛₑ can be based at least in part on detecting an undocking event between a wearable device and a docking device (e.g., indicating, in some cases, that the user is finished sleeping and has decided to put their wearable device on before or after leaving the bed). The enter bed time t_{bed} time for a second, subsequent sleep session can also be defined based on a rise threshold duration (e.g., the user has left the bed for at least 3 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based on the system monitoring the user's sleep behavior. In some cases, the threshold period can be set and/or overridden by detection of a docking or undocking event.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, referring to the timeline 301 of FIG. 3, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 4 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

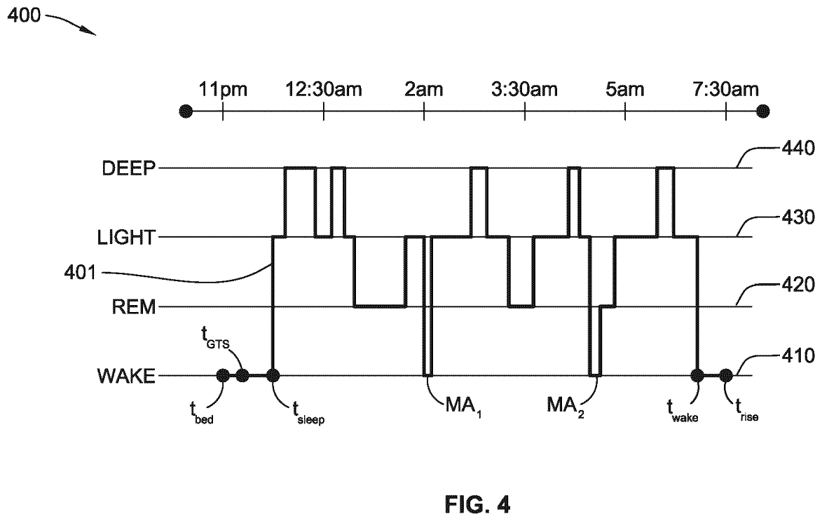

Referring to FIG. 4, an exemplary hypnogram 400 corresponding to the timeline 301 (FIG. 3), according to some implementations, is illustrated. As shown, the hypnogram 400 includes a sleep-wake signal 401, a wakefulness stage axis 410, a REM stage axis 420, a light sleep stage axis 430, and a deep sleep stage axis 440. The intersection between the sleep-wake signal 401 and one of the axes 410-440 is indicative of the sleep stage at any given time during the sleep session.

The sleep-wake signal 401 can be generated based on physiological data associated with the user (e.g., generated by one or more of the sensors 130 described herein). The sleep-wake signal can be indicative of one or more sleep states, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 400 is shown in FIG. 4 as including the light sleep stage axis 430 and the deep sleep stage axis 440, in some implementations, the hypnogram 400 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration ratio, a number of events per hour, a pattern of events, or any combination thereof. Information describing the sleep-wake signal can be stored in the memory device 114.

The hypnogram 400 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (t_{GTS}) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 4), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 4 minutes, greater than about 10 minutes, etc.)

The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 4), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM stage) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 130 can be used to determine or identify the enter bed time (t_{bed}) (e.g., via detection of a docking event), the go-to-sleep time (t_{GTS}) (e.g., via detection of a docking event), the initial sleep time (tₛₗₑₑₚ), one or more first micro-awakenings (e.g., MA₁ and MA₂), the wake-up time (t_{wake}) (e.g., via detection of an undocking event), the rising time (tᵣᵢₛₑ) (e.g., via detection of an undocking event), or any combination thereof, which in turn define the sleep session. For example, the enter bed time t_{bed} can be determined based on, for example, data generated by the motion sensor 138, the microphone 140, the camera 150, a detected docking event, or any combination thereof. The go-to-sleep time can be determined based on, for example, data from the motion sensor 138 (e.g., data indicative of no movement by the user), data from the camera 150 (e.g., data indicative of no movement by the user and/or that the user has turned off the lights) data from the microphone 140 (e.g., data indicative of the using turning off a TV), data from the user device 170 (e.g., data indicative of the user no longer using the user device 170), data from the pressure sensor 132 and/or the flow rate sensor 134 (e.g., data indicative of the user turning on the respiratory device 122, data indicative of the user donning the user interface 124, etc.), data from the wearable device 190 (e.g., data indicative that the user is no longer using the wearable device 190, or more specifically, has docked the wearable device 190 with the docking device 192), data from the docking device (e.g., data indicative that the user has docked the wearable device 190), or any combination thereof.

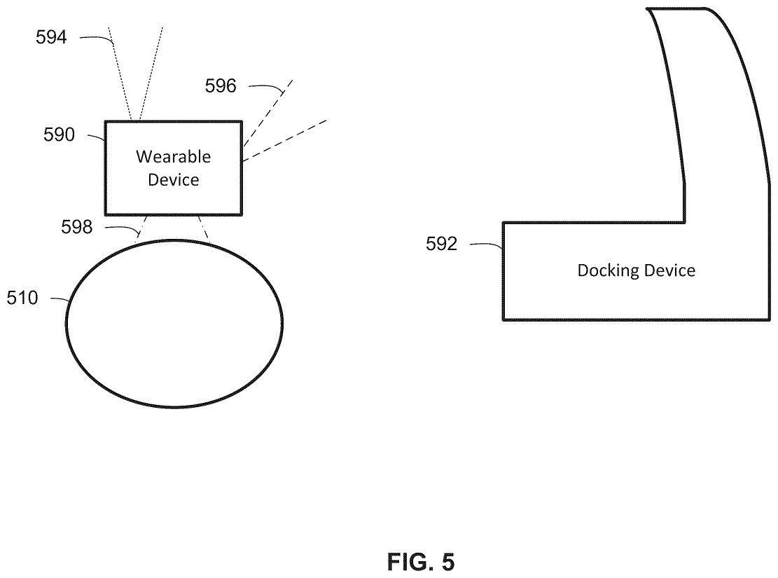

FIGS. 5-9 relate to facilitating collection of physiological data by automatically changing sensor configurations in response to detection of a docking event between a wearable device (e.g., wearable device 190 of FIG. 1) and a docking device (e.g., docking device 192 of FIG. 1).

Examples of wearable devices include smartwatches, fitness trackers, earbuds, headphones, AR/VR headsets, smart glasses, smart clothing, smart accessories (e.g., smart jewelry), and the like. Examples of docking devices include device stands or cradles (e.g., watch stands), charging mats, battery packs (e.g., battery packs for charging smartphones and accessories), other electronic devices (e.g., smartphones capable of providing power to a peripheral, such as via a wireless connection), and the like. Docking devices can be mains-powered (e.g., connected to a building's or site's power, such as via an electrical outlet or a hardwire connection), battery powered, or otherwise powered (e.g., solar powered or wind powered).

Generally, when the wearable device docks with the docking device, the wearable device and docking device establish i) a physical connection (e.g., a feature of the wearable device resting in a corresponding detent of the docking device or a magnetic attraction); ii) a power connection (e.g., via a wireless power coupling or a wired connection); iii) a data connection (e.g., via a wireless data connection or a wired connection); or iv) any combination of i-iii. In some cases, the wearable device can dock with the docking device by a wireless connection (e.g., a QI wireless connection or a near field connect (NFC) wireless connection) or a wired connection (e.g., a USB or USB-C connection, a lightning connection, a proprietary connection, or the like). In some cases, the docking device may be a smart device, such as a smartphone. In other cases, the docking device may be a charging device, such as a charging mat for a smartphone, and which may be configured to be able to dock with a wearable device and/or a respiratory therapy device, and a smartphone or other smart device, at the same time.

The wearable device and docking device can define a wearable system that can include one or more sensors on the wearable device, and optionally one or more sensors on the docking device. In some cases, additional devices (e.g., additional wearable devices, additional docking devices, additional user devices) can also be used, in which case one or more sensors of the additional devices may be used as well.

The wearable device (and docking device, and more generally the wearable system) can operate in a plurality of modes, such as a worn mode (e.g., a mode in which the wearable device is being worn by a user and otherwise operating normally), a worn power-saving mode (e.g., a mode in which the wearable device is being worn by a user and operating with reduced power usage to preserve the wearable device's battery), a docked mode (e.g., a mode in which the wearable device is docked with a docking device and otherwise operating normally), and a docked power-saving mode (e.g., a mode in which the wearable device is docked with a docking device and operating with a reduced power usage to preserve the docking station's power source). In some optional cases, a wearable device can be in a worn and docked mode, in which case the wearable device is being worn by the user but still receiving power from a nearby docking station (e.g., via an extended-distance wired connection or an extended-distance wireless connection).

In each of these different modes, the wearable device can use a specific sensor configuration defined for that mode. A sensor configuration includes a set of sensors (e.g., one or more sensors) used and/or a set of sensing parameters used for the set of sensors. The set of sensors can define which sensors are used to acquire data while a particular mode is active. The sensing parameters can define how each of the set of sensors is driven, accessed, or otherwise interacted with, or how the sensor data is preprocessed (e.g., denoising, normalizing, or other preprocessing). For example, sensing parameters can define a sampling rate, a sampling depth, a gain, any other suitable adjustable parameter for making use of a sensor, or any combination thereof. As another example, sensing parameters can define which preprocessing techniques are used to preprocess the sensor data and/or what settings are used for each of the preprocessing techniques. In some cases, the sensing parameters only include those sensing parameters that are different than a default sensing parameter.

In response to a docking event or an undocking event, the wearable device (or docking device or more generally the wearable system) can automatically switch modes. A docking event is when a wearable device becomes docked with the docking device, and an undocking event is when the wearable device becomes undocked with the docking device. Docking events can be defined by i) establishment of a physical connection; ii) establishment of a power connection; iii) establishment of a data connection; iv) or any combination of i-iii. Likewise, undocking events can be defined by i) uncoupling of a physical connection; ii) breaking of a power connection; iii) breaking of a data connection; iv) or any combination of i-iii. In some cases, docking and undocking events can be defined manually (e.g., by the user pressing a “docked” or “undock” button).

In some cases, a particular docking event can be confirmed or otherwise informed by additional sensor data. For example, a wearable system can be established to enter a first type of docked mode when the wearable device is docked with a first docking device in the user's kitchen, but enter a second, different type of docked mode when the wearable device is docked with a second docking device in the user's bedroom. In such cases, sensor data can be used to determine to which docking device the wearable device is docked. For example, environmental data acquired by the wearable device can be used to generate a prediction about the location of the wearable device (e.g., in the kitchen or in the bedroom) at the time of the docking event. Likewise, environmental data acquired by the docking device can be used to confirm that the wearable device is being docked with that particular docking device (e.g., the wearable device and docking device are obtaining similar readings for ambient light levels and/or ambient sound levels). In some cases, the wearable system can establish a location fingerprint for the location of a docking device and/or other locations. Each location fingerprint can be a unique set of location-specific characteristics (e.g., sounds, acoustic reflection patterns, RF background noise, LIDAR or RADAR point clouds, and the like) that are discernable by sensor data collected by the wearable device and/or docking device. As another example, wireless signal levels (e.g., signal levels of nearby wireless access points) can be used to help identify that the wearable device being docked is in the same location as a particular docking device. In some cases, however, the docking device can merely provide identifying information to the wearable device via a data connection. In some cases, a Bluetooth wireless signal can be used to identify whether the wearable device is positioned near a desired docking device, and/or positioned in a certain environment (e.g., a bedroom or a kitchen). The Bluetooth wireless signal can include an active data link between the wearable device and the docking device, although that need not always be the case. In some cases, the Bluetooth wireless technology could be used to merely identify when the wearable device is within a certain distance of the docking device. In some cases, the Bluetooth connection can be between the wearable device and a device other than the docking device, such as a television, a smart light, a smart plug, or any other suitable Bluetooth-enabled device.

In some cases, activity information from a user device (e.g., a smartphone) or another wearable device can be used to confirm that a docking event has occurred. For example, if the activity information from the user's smartphone shows that the user is lying in bed using their phone, has put their phone down, or has started charging their phone, an assumption can be made that the wearable device is indeed being docked (e.g., docked for a sleep session). Likewise, if the activity information from the user's smartphone shows that the user is walking around or actively engaged in an activity (e.g., playing a game, watching a movie, engaging in a workout), an assumption can be made that the wearable device is not intended to be docked or is only temporarily docked.

Generally, when a wearable device becomes docked, it will receive power from the docking device. Thus, there is no longer a need to preserve battery life, and the set of sensors used and/or the sensing parameters used can be selected to maximize or emphasize fidelity of the data collected rather than having to balance fidelity with power usage. Likewise, when a wearable device becomes undocked, it no longer receives power from the docking device, and thus must go back to balancing fidelity with power usage.

In some cases, when a wearable device becomes docked, the wearable system can leverage sensors included in the docking device, which may be more powerful, better positioned, more capable (e.g., a different and more precise sensing method), or otherwise more desirable to use (e.g., to avoid extra wear on sensors of the wearable device) as compared to similar or corresponding sensors of the wearable device. For example, while a wearable device may make use of motion sensors to detect a user's biomotion while the wearable device is being worn, such motion sensors may be unsuitable to detect the user's biomotion when the wearable device is docked. Thus, in response to docking the wearable device, the docking station may automatically start collecting SONAR or RADAR sensor data to detect the user's biomotion (e.g., an acoustic biomotion sensor as described here). As another example, smaller RADAR sensors and/or acoustic sensors on a wearable device may induce artifacts in the collected data, whereas larger versions of the same sensors on a docking device may be able to collect the data with reduced or no artifacts.

In some cases, when a wearable device becomes docked, it can pass processing duties to another device, such as to a processor in the docking device and/or a processor communicatively coupled (e.g., via a wired or wireless network) to the docking device. In such cases, any sensor data collected by the wearable device while docked can be passed to the docking device. In some cases, however, when the wearable device becomes docked, it can continue some or all data processing duties. In such cases, any sensor data collected by the docking device or other external sensors can be passed to the wearable device for processing.

In some cases, the docking device can also be used to improve performance of one or more sensors of the wearable device when the wearable device is docked with the docking device. For example, the docking device can resonate, amplify, or redirect signals to the sensor(s) of the docked wearable device.

In some cases, the docking device can improve a position of a sensor (e.g., a line-of-sight sensor) of a wearable device. In some cases, the wearable sensor can include instructions for where to place the docking device and/or wearable device to achieve desired results. In some cases, the docking device can manually or automatically reposition the wearable device to achieve desired results. In some cases, an initial setup test can include having the user lay in a usual position in bed and test different positions of the docking station and/or wearable device until desired results are achieved. In some cases, the wearable device can include a visual cue (e.g., an arrow on the housing of the wearable device or a digital icon on a digital display of the wearable device) that indicates how to position and/or orient the wearable device. In some cases, feedback can be provided (e.g., visual and/or audio feedback) as the user changes the position and/or orientation of the wearable device, permitting the user to find the correct placement to achieve desire results. In some cases, this feedback can be an indication of the user's breathing pattern, which can be used to determine whether or not the wearable device and/or docking device can adequately sense the user's breathing.

In use, the wearable system is able to leverage sensor data from both before and after the wearable device becomes docked and/or undocked with a docking station. In some cases, the act of docking or undocking the wearable device can also provide additional information that can be leveraged, such as to identify an approximate time in bed or rise time.

In some cases, sensor data collected in one mode can be used to calibrate sensor data collected in another mode. For example, sensor data collected for several sleep sessions while the user is wearing the wearable device can be used to calibrate sensor data collected while the wearable device is docked. In such an example, one or more parameters (e.g., sleep-related parameters) that are determined using the sensor data collected while the wearable device is being worn can be compared with one or more parameters that are determined using the sensor data collected while the wearable device is docked. The sensor data collected while the wearable device is being docked can be adjusted such that the one or more parameters derived therefrom match expected values for the one or more parameters based on the sensor data collected while the wearable device is being worn. In some cases, calibration can go in a reverse direction, with sensor data from the wearable device while docked being used to calibrate the sensor data from the wearable device while being worn.

In some cases, calibration can occur especially using sensor data acquired close to a docking or undocking event (e.g., transitional sensor data). This transitional sensor data can be especially useful since the same physiological parameters may be able to be measured using different means (e.g., according to the different modes) at around the same time. For example, heartrate measured by the wearable device while being worn can be compared to heartrate as measured by the docking device when the wearable device is docked. Since the heartrate is not expected to change significantly in a short period of time, the comparison between the two techniques for measuring heartrate can be used to calibrate sensor data (e.g., the sensor data from the docking station).

In some modes, such as an example docked mode, collection of sensor data can established such that it is triggered by external sensors (e.g., external motion detectors). In such an example, the wearable system will wait until a trigger is received (e.g., motion is detected by a separate motion detector) before beginning to collect sensor data.

In some modes, such as an example docked mode, collection of sensor data from certain sensor(s) and/or using certain sensing parameters can be performed only after being triggered by a detected physiological parameter. For example, a low-power and/or unobtrusive sensor can periodically sample to detect an apnea. In response to the detected apnea, additional sensors can be used and/or additional sensing parameters can be used to acquire higher-resolution data for a duration of time following the apnea, in the hopes of acquiring more informative data associated with any subsequent apneas in the same cluster as that first apnea. In another example, certain low-power sensors and/or sensing parameters can be used while it is determined that the user is in a first sleep state, whereas different sensors and/or different sensing parameters can be activated to acquire higher-resolution data when it is determined that the user is in a second sleep state.

In some cases, one or more sensors of the wearable device and one or more sensors of the docking device can be used in combination to provide multimodal sensor data usable to determine a physiological parameter. For example, a PPG sensor on a wearable device can be used in concert with an acoustic-based (e.g., SONAR) or RADAR-based biomotion sensor to identify OSA events and/or discern OSA events from CSA events.

In some cases, detection of a docking event or undocking event can automatically trigger another action, such as automatically trigger one or more lights to dim or go off, automatically trigger playing of an audio file, or perform other actions.

In some cases, detection of a docking event or an undocking event can trigger a change in processor speeds of one or more processors in the docking device, wearable device, and/or respiratory therapy device, etc. Additionally, or alternatively, the detection may trigger use of more or fewer cores (e.g., central processing unit (CPU)) cores by the docking device, wearable device, and/or respiratory therapy device, etc. In some cases, the detection may trigger activation/de-activation of artificial intelligence (AI) processing (e.g., via an AI accelerator chip). In these examples, the detection of a docking event or an undocking event allows the docking device, wearable device, and/or respiratory therapy device, etc. to optimize electrical power and/or processing power depending on how the respective device is being used at the time.

In some cases, since many wearable devices are normally designed for healthy individuals, the fusion of sensor data available using the disclosed wearable system can provide more accurate sleep hypnograms and other physiological parameters for individuals with sleep disordered breathing or other disorders. These more accurate physiological parameters are enabled by the fusion of sensor data collected by a wearable device when being worn while awake, sensor data collected by a wearable device when being worn while asleep, and sensor data collected by the wearable system while the wearable device is docked to a docking device while asleep. For example, a principal component analysis can be performed between multiple sensors to ensure more accurate results between modes (e.g., more accurate results between sensors of the wearable device and sensors of the docking device).

In some cases, activating a mode in response to a docking event or undocking event can include engaging in a delay. For example, when a wearable device is docked to a docking station, a preset delay (e.g., seconds, minutes, tens of minutes, hundreds of minutes, and the like) can be taken to avoid collecting sensor data while the user is preparing to go to sleep.

In some cases, an autocalibration system can be implemented. The autocalibration system can involve acquiring sensor data while the user performs certain predefined actions, such as speaking in a normal voice while in bed (e.g., to check a microphone), performing a deep breathing exercise (e.g., to ensure loud breathing can be heard), and the like. In some cases, an acoustic signal (e.g., an inaudible sound) and/or RADAR (e.g., FMCW, pulsed FMCW, PSK, FSK, CW, UWB, pulsed UWB, white noise, etc.) signal can be emitted to detect movements of the user's chest while the user is engaging in deep breathing. In some cases, the autocalibration system can detect perturbations during speech. The sensor data acquired during the autocalibration process can be used to calibrate and/or otherwise adjust sensor data being acquired from the one or more sensors of the wearable device and/or the docking device.

In some cases, collected sensor data from a wearable system can be used to improve compliance with respiratory therapy, such as via detecting the sounds of air leaks and/or a user snoring and merging such data with data from the respiratory therapy device. This merged data can be useful to identify benefits of respiratory therapy compliance, which can help improve the user's own respiratory therapy compliance. In some cases, the collected sensor data is from a wearable system presenting an entrainment stimulus to the user based at least in part on the entrainment signal.

Sensor data acquire in a first mode can be synchronized with sensor data acquired in a second mode. Synchronizing the sensor data across different modes can include synchronizing sensor data from different sensors of the same type, different types of sensors, and the same sensors operating under different sensing parameters.

In some cases, different sensor data can be applied different weighting depending on the underlying sensor's expected fidelity and/or that sensor's signal-to-noise ratio. For example, while acoustic data can be acquired simultaneously by a microphone in the wearable device and a microphone in the docking device, the sensor in the docking device may be a larger and more robust sensor capable of higher fidelity, in which case a higher weighting value will be applied to the sensor data from the docking device than to the sensor data from the wearable device. In some cases, weighting values can change dynamically, such as when a particular sensor is expected to achieve an overall higher accuracy.

In some cases, a docking device can be coupled to and/or incorporated in a respiratory therapy device. In some cases, the wearable device can leverage one or more sensors of the respiratory therapy device when docked. In some cases, the physiological parameters determined by the wearable device when docked can be used to adjust one or more parameters of the respiratory therapy device. In some cases, the wearable device can operate as a display for the respiratory therapy device (e.g., via connecting corresponding application programming interfaces (APIs) at a cloud level and/or otherwise sharing data). In some cases, the collected sensor data from a docking device, and/or from a wearable device, may be used to facilitate or augment a program to help improve a person's sleep (e.g., via a sleep therapy plan such as a CBT-I program) and/or to become habituated with a respiratory therapy system (e.g., via a respiratory therapy habituation plan that allows a new user to become familiar with the respiratory therapy system, breathing pressurized air, reducing anxiety, etc.). For example, the docking device may present a breathing entrainment stimulus, such as a light and/or sound signal, to a user based at least in part on a sensed respiratory signal of the user. Other sensed signals of the user may include heart rate, heart rate variability, galvanic skin response, or a combination thereof. An entrainment program may encourage the user's breathing pattern, via the breathing entrainment stimulus, towards a predetermined target breathing pattern (such as a target breathing rate) which has been predicted, or has been learned for that user, to result in the user achieving (i) a sleep state, either within any time period or within a predetermined time period, (ii) breathing (optionally with confirmed breathing comfort via subjective and/or objective feedback) of pressurized air from a respiratory therapy system at prescribed therapy pressures, or (ii) i and ii.

In some cases, a docking device can be configured to allow docking by a respiratory therapy device. The docking device can thus be used to power the respiratory therapy device during use, e.g., when supplying pressurized air to a user, or to charge the respiratory therapy device having a power storage facility, e.g., a battery. In cases in which the respiratory therapy device has a power storage facility, such as a battery, the respiratory therapy device may be comprised in a respiratory therapy system wearable by the user, such as wearable about the head and face of the user. Thus, prior to (and/or after) use of such a respiratory therapy system, the respiratory therapy device may be charged when docked with the docking device. Docking to the docking device may also allow data, such as respiratory therapy use data, physiological data of the user, etc., to be transferred from the respiratory therapy device via wired or wireless means to the docking device and processed locally and/or transmitted to a remote location, e.g., to the cloud, and optionally displayed to the user or a third party such as a physician.

In some cases, certain sensors can be automatically disabled or prohibited when the wearable system is in a first mode, but enabled or allowed when the wearable system is in a second mode. For example, to protect privacy, a microphone or other sensor in the wearable device can be disabled or prohibited while it is worn, but can be enabled or allowed (e.g., to detect, optionally for recording, speech, respiration, or other data) when the wearable device is docked, or vice versa.

In some cases, sensor data collected from the wearable device while being worn can be compared with sensor data collected from the wearable device when docked to obtain transitional sensor data. The transitional sensor data can include sensor data associated with transitions between a docked and undocked state. For example, temperature data acquired from the wearable device while worn can be compared with temperature data acquired from the wearable device while docked to determine how long it takes for the temperature to drop from body temperature to ambient temperature, which information can be leveraged to determine physiological parameters.

In some cases, the specific sensors used in a docked mode can depend on the capabilities of the docking device. In such cases, the wearable device can automatically or manually (e.g., via user input) obtain capability information associated with the docking device (e.g., a listing of available sensors and/or available sensing parameters). In some cases, the docking device can provide identification information and/or capability information directly to the wearable device, such as via a data connection. In other cases, the wearable device can determine identification information associated with the docking device from sensor data (e.g., from camera data), which can be used to determine capability information associated with the identification information (e.g., via a lookup table). Depending on the docking device's capability information, the specific sensors and/or sensing parameters used in a given mode can be selected.

In some cases, charging circuitry in the wearable device and/or in the docking device can automatically adjust a charging rate to maintain a safe temperature within the wearable device and/or within the docking device. In some cases, the charting circuitry can adjust the charging rate based at least in part on the sensor configuration for the mode in which the wearable system is operating. For example, when certain sensors are being used that generate a noticeable amount of heat, the charging circuitry may automatically charge the battery at a lower rate to avoid overheating. However, if a different set of sensors and/or different sensing parameters are being used that would generate less heat, the charging circuitry may automatically charge the battery at a higher rate.

In some cases, the wearable device makes use of at least one contacting sensor when worn and makes use of at least one non-contacting sensor when docked with a docking device. In some cases, the wearable device makes use of at least one line-of-sight sensor (e.g., a LIDAR sensor) and at least one non-line-of-sight sensor (e.g., a microphone to detect apnea events).

In some cases, sensor data collected while the wearable device is being worn by the user can help identify a user's state before going to sleep. For example, physiological data associated with the user just prior to docking the wearable device with the docking device can indicate that the user is in a state of hyper-arousal at a time when the user is planning to go to sleep. In response to detecting that hyper-arousal, the system can automatically present a notification to the user, such as a notification instructing the user to perform a calming meditation, perform deep breathing, or do a different activity for a while before attempting to go to sleep.

In an example use case, a wearable device that is a smartwatch can be used by a user throughout the day, collecting information about the user's activity level and/or other physiological data associated with the user (e.g., via motion sensors and PPG sensors). When the user gets ready to go to sleep, the user can place the smartwatch on a corresponding charging stand, which automatically causes the smartwatch to begin capturing acoustic signals (e.g., via a microphone or acoustic sensor), which can be used to determine the user's biomotion during a sleep session, which can further be used to determine sleep stage information and other sleep-related physiological parameters. Then, when the user wakes up in the morning and removes the smartwatch to wear it again, the smartwatch can automatically switch back to collecting information about the user's activity level and/or other physiological data. The combination of sensor data acquired before, during, and/or after the sleep session can be used to provide information and insights about the user. In some cases, the sensor data acquired before the sleep session (e.g., average resting heart rate throughout the day or motion data throughout the day) can be used with the sensor data acquired during the sleep session to determine a physiological parameter (e.g., a more accurate determination of sleep stage based on biomotion). In some cases, the sensor data acquired before the sleep session can be used with sensor data acquired during the sleep session to help diagnose and/or treat a sleep-related or respiratory-related disorder, such as by generating an objective score associated with the severity of the disorder.

In another example use case, if a wearable device detects heart-related issues (e.g., atrial fibrillation) while being worn during a day, the wearable system can automatically trigger advanced heartrate detection, making use of more robust sensors and/or sensing parameters, when the wearable device is docked at night.

In another example use case, actimetry and heart rate can be captured by smartwatch when on wrist of user, and at night, RF and/or sonar sensors in a smartwatch cradle can be leveraged to capture the same, similar, or equivalent data.

In another example use case, the wearable device can collect periodic audio data throughout the day while being worn. This periodic audio data can be used to detect certain keywords, particular speech patterns, confusion levels in speech, stutters, gaps, and the like. When the wearable device is docked at night, audio data can be collected (e.g., from one or more sensors of the wearable device and/or the docking device) to detect respiration sounds to find apneic gaps or to detect other sleep-related physiological parameters. In such cases, since the wearable device is docked, higher data rates can be used (e.g., collecting audio data more often than when the wearable device was being worn) to detect OSA events with higher fidelity. In some cases, if the system detects a low confidence of an OSA risk on a first night, it can ask the user to opt in for higher-resolution data processing for a subsequent night in the hopes of detecting the user's OSA risk with a higher level of confidence.

FIG. 5 is a schematic diagram depicting a wearable device 590 operating in a first mode, according to certain aspects of the present disclosure. The wearable device 590 can be any suitable wearable device, such as wearable device 190 of FIG. 1. In some cases, the wearable device 590 is a smartwatch, such as the depiction of wearable device 190 in FIG. 2. The docking device 592 can be any suitable docking device, such as docking device 192 of FIG. 1. In some cases, the docking device 592 is a smartwatch stand, such as the depiction of docking device 192 in FIG. 2. The wearable device 590 can be battery powered.

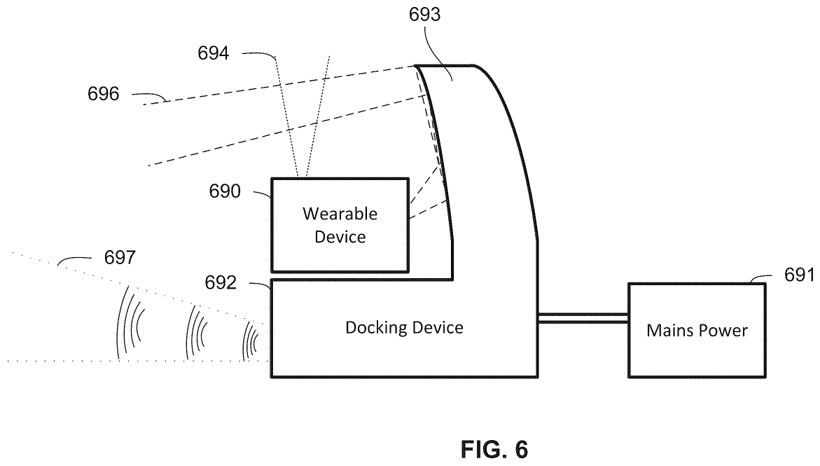

Wearable device 590 can collect sensor data using one or more sensors (e.g., one or more sensors 130 of FIG. 1). When the wearable device 590 is being worn, such as on a wrist 510 of a user, the wearable device 590 may generally operate in a first mode. The first mode (e.g., worn mode) can make use of a first sensor configuration. The first sensor configuration can include a set of sensors used to collect sensor data and a set of sensing parameters used to operate the set of sensors. As an example, in the first sensor configuration, the wearable device 590 may collect blood oxygenation signals 598 via a PPG sensor, may collect acoustic signals 596 via a microphone, and may collect light signals 594 via a camera or other light sensor. In the first sensor configuration, the wearable device 590 may operate each of these sensors using sensing parameters selected to preserve battery life while still achieving adequate performance. For example, the light signals 594 may be captured by using a relatively low sampling rate (e.g., 1 Hz) to preserve battery life while the wearable device 590 is operating in the first mode. However, once the wearable device 590 begins operating in a different mode (e.g., second mode, as described herein with reference to FIG. 6), the light signals 594 may be captured using a different sampling rate, such as a relatively high sampling rate (e.g., 100 Hz). Similarly, the microphone may collect the acoustic signals 596 using a first set of sensing parameters while in the first mode (e.g., a certain sampling rate, a certain bit depth, and the like) and may operate using a different set of sensing parameters while in another mode (e.g., a higher sampling rate, a higher bit depth, and the like).

While operating in the first mode, the wearable device 590 is not docked to the docking device 592.

FIG. 6 is a schematic diagram depicting a wearable device 690 operating in a second mode while docked with a mains-powered docking device 692, according to certain aspects of the present disclosure. Wearable device 690 and docking device 692 can be any suitable wearable device and docking device, such as wearable device 590 and docking device 592 of FIG. 5, respectively.

Docking device 592 can be connected to mains power 691 (e.g., a building power, such as via an electrical socket or a hardwired connection) permanently or removably. The wearable device 690 is depicted as being docked with the docking device 692. When docked, the wearable device 690 can receive power from the docking device 692, such as via a wireless power connection (e.g., inductive power transfer, such as the Qi standard or a near field connect (NFC) standard) or via a wired connection (e.g., such as via exposed electrodes). In some cases, the wearable device 690 can also exchange data with the docking device 692.

While docked, the wearable device 690 can operate in a second mode (e.g., a docked mode). In the second mode, the wearable device 690 can automatically use a second sensor configuration that is different than the first sensor configuration (e.g., the first sensor configuration described with respect to FIG. 5). The second sensor configuration can use different sensors than those in the first sensor configuration, such as fewer sensors, additional sensors, or alternate sensors. In the second sensor configuration, the sensors that are used can be operated using sensing parameters that are different than those of the first sensor configuration.

In an example where different sensors are used, while wearable device 590 of FIG. 5 collected light signals 594 via a camera or other light sensor, wearable device 690 collects light signals 694 via a different camera or different light sensor. The different camera or different light sensor can be preferable to be used while the wearable device 690 is docked, such as if it requires more power to operate or performs poorly when the wearable device 690 is being worn (e.g., if the sensor performs poorly when undergoing movement characteristic of a worn wearable device 690 or if the sensor performs poorly when positioned next to the heat of the user's body).

In an example where the same sensors are used, while wearable device 590 of FIG. 5 collected light signals 594 via a camera or other light sensor, wearable device 690 collects light signals 694 via the same camera or other light sensor being operated using different sensing parameters. The sensing parameters of the wearable device 590 of FIG. 5 may include capturing the light signals 594 at a sampling rate of 1 Hz. However, the sensing parameters of the wearable device 690 may include capturing the light signals 694 at a sampling rate of 100 Hz. Since the wearable device 690 is receiving power from the docking device 692, the increased power requirements of using such a high sampling rate 100 Hz are without concern.

In some cases, a docking device 692 can optionally include a reflector 693 designed to reflect signals towards a sensor of the wearable device 690. For example, while wearable device 590 of FIG. 5 collected acoustic signals 596 by generally exposing a microphone to an environment, wearable device 690 collects acoustic signals 696 by exposing a microphone to a reflector 693 that redirects the acoustic signals 696 from a specific region in front of (e.g., or to a side of) the docking device 692. As depicted in FIG. 6, the acoustic signals 696 directed towards the docking device 692 from the left side of the page are redirected by the reflector 693 towards a corresponding microphone of the wearable device 690. While described with reference to acoustic signals 696, the reflector 693 can be configured for use with any suitable signals (e.g., RF signals or other electromagnetic signals). In some cases, the reflector 693 can be manually or automatically adjustable to ensure the desired acoustic signals 696 are being capture.

In some cases, docking device 692 can include a speaker for outputting sound 697 (e.g., sonic sound, ultrasonic sound, infrasonic sound). For example, when the wearable device 690 is docked with the docking device 692, the docking device 692 may automatically begin outputting sound 697, which can be reflected off objects in the environment (e.g., the body of a user) and captured as acoustic signals 696. The use of a speaker within the docking device 692 instead of a speaker in the wearable device 690 can extend the lifespan of the speaker within the wearable device 690 (e.g., avoid overuse) and, in some cases, can permit different sounds to be generated that may otherwise be limited by the size of the speaker within the wearable device 690.

In some cases, the docking device 692 can be shaped to promote having one or more sensors of the wearable device 690 face a desired direction. For example, a docking device 692 that is a watch stand can support a wearable device 690 that is a smartwatch in such a fashion that its microphone is pointed at the reflector 693 or pointed at a user when the docking device 692 is positioned in an expected position on a user's nightstand (e.g., with the watch face facing the user). In another example, the docking device 692 can be designed to lift the wearable device 690 to a suitable height to permit certain sensors (e.g., line-of-sight sensors) to collect data from the user. For example, a watch stand intended for use on a nightstand may have a height designed to raise the smartwatch sufficiently off the nightstand to achieve a good line-of-sight to a user. Such a height can be manually or automatically adjustable, or can be preset based on average heights of nightstands and beds.

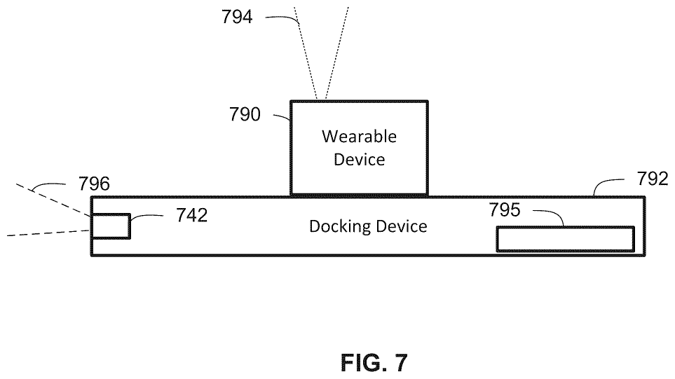

FIG. 7 is a schematic diagram depicting a wearable device 790 operating in a second mode while docked with a battery-powered docking device 792, according to certain aspects of the present disclosure. Wearable device 790 and docking device 792 can be any suitable wearable device and docking device, such as wearable device 190 and docking device 192 of FIG. 1, respectively. As depicted in FIG. 7, docking device 792 is a battery-powered docking device, such as a smartphone, another user device, or a battery pack. Docking device 792 can include a battery 795.

Wearable device 790 can dock to docking device 792 as described herein, such as via magnetic coupling (e.g., magnetic physical coupling and magnetic power coupling). When a battery-powered wearable device 790 is used, the mode used by the wearable device 790 and/or docking device 792 can depend on the amount of charge remaining in the battery 795. For example, when the battery 795 is fully charged, the wearable device 790 and/or docking device 792 can operate in a standard docking mode (e.g., similar to the second mode described with reference to wearable device 690 of FIG. 6). However, when the battery 795 is below a threshold charge, the wearable device 790 and/or docking device 792 can enter a power-saving mode, which can be similar to the first mode described with reference to wearable device 590 of FIG. 5 or another mode.

As depicted in FIG. 7, in the second mode, the wearable device 790 collects light signals 794 via a camera or other light sensor, while the docking device 792 collects acoustic signals 796 via microphone 742. The microphone 742 of the docking device 792 can be a more robust and/or higher-quality microphone than that of the wearable device 790.

In some cases, the wearable device 790 can establish a data connection with the docking device 792, such as to share charge information of the battery 795, share capability information of the docking device 792 (e.g., what sensors are available for use), share sensor data, and/or share other data.

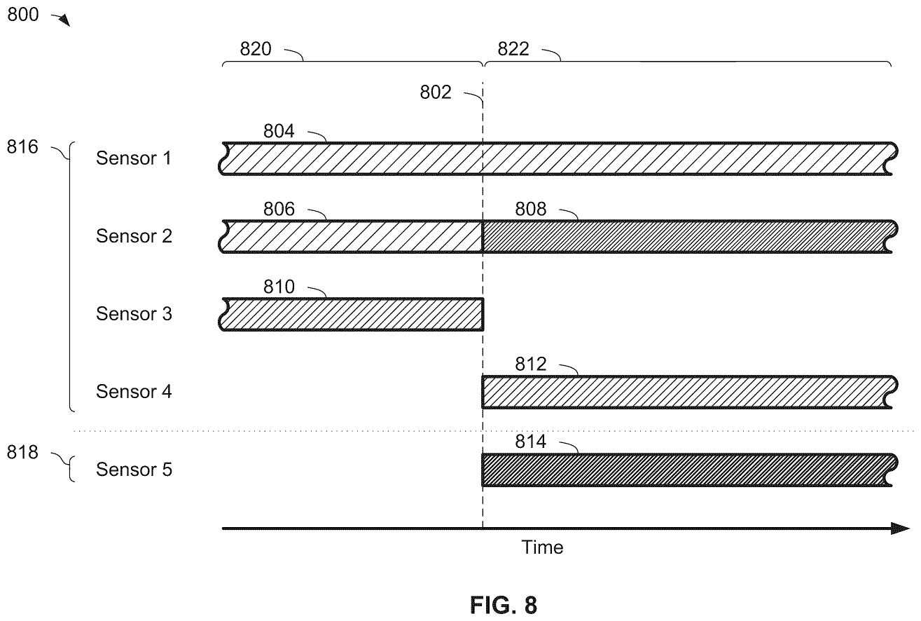

FIG. 8 is a chart 800 depicting sensor configurations before and after a docking event, according to certain aspects of the present disclosure. The sensor configurations can represent sensor configurations used by a wearable device and optionally a docking device. Any suitable wearable device and docking device can be used, such as wearable device 590 and docking device 592 of FIG. 5. Any suitable sensors may be comprised in the wearable device and/or the docking device. For example, the wearable device and/or the docking device may comprise a camera for light (e.g., still images, video images, etc.) and/or thermal imaging. The sensors in the wearable device and the docking device are not particularly limited and the respective sensors may be the same (e.g., substantially identical), of the same type (e.g., the same functionality), or may be different but generate substantially the same type of data.

The wearable device can include a set of sensors 816 that includes Sensor 1, Sensor 2, Sensor 3, and Sensor 4, each of which can be any suitable type of sensor. The docking device can include a set of sensors 818 that includes Sensor 5, which can be any suitable type of sensors. Any number of sensors and types of sensors can be used in either set of sensors 816, 818.

Chart 800 depicts the time before and during a single sleep session, specifically the time before and after a docking event 802. Before the docking event 802, the wearable device can operate using a first sensor configuration which involves collecting sensor data 804, sensor data 806, and sensor data 810. Sensor data 804 is collected from Sensor 1 using a first set of sensing parameters for Sensor 1. Sensor data 806 is collected from Sensor 2 using a first set of sensing parameters for Sensor 2. Sensor data 810 is collected from Sensor 3 using a first set of sensing parameters for Sensor 3.

Upon detection of the docking event 802, the wearable device (and docking device) can operate using a second sensor configuration 822. In the second sensor configuration 822, sensor data 804, sensor data 808, sensor data 812, and sensor data 814 can be collected. In the second sensor configuration 822, sensor data 804 can continue to be collected from Sensor 1 using the same first sensing parameters for Sensor 1. Sensor data 808 can be collected from Sensor 2, but using second sensing parameters for Sensor 2. Sensor data 812 can be collected from Sensor 4, which was unused in the first sensor configuration 820. Sensor data 814 can be collected from Sensor 5.

For illustrative purposes, the intensity of the fill within the bars indicating sensor data is indicative of power usage (e.g., watts, or energy per unit time). For example, sensor data 808 requires more power than sensor data 806, even though acquired from the same Sensor 2. Likewise, sensor data 808, sensor data 812, and sensor data 814 all require more power than sensor data 804 and sensor data 806. As depicted in chart 800, it is clear that the use of different modes with concomitant sensor configurations permits more power-hungry sensors and/or sensing parameters to be used when the wearable device is docked, and thus receiving power from the docking device.

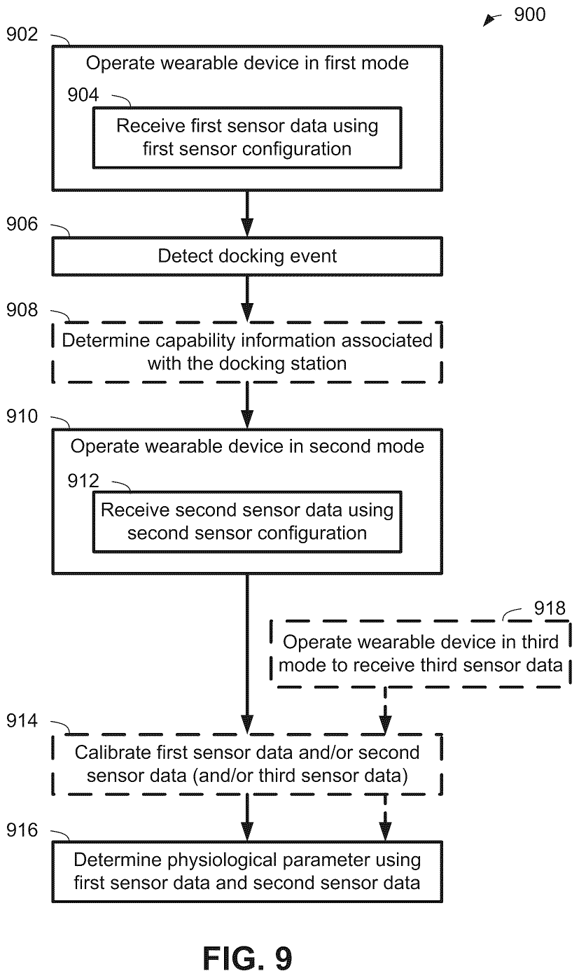

FIG. 9 is a flowchart depicting a process for automatically switching modes of a wearable device in response to detecting a docking event, according to certain aspects of the present disclosure. Process 900 can be performed by system 100 of FIG. 1, such as by a wearable device (e.g., wearable device 190 of FIG. 1) and a docking device (e.g., docking device 250 of FIG. 2).

At block 902, the wearable device can be operated in a first mode. Operating the wearable device in a first mode can include receiving first sensor data at block 904. Receiving first sensor data at block 904 can include using a first sensor configuration. The first sensor configuration can define a first set of sensors (e.g., one or more sensors) of the wearable device that are used for collecting sensor data, and/or define a first set of sensing parameters used to collect the sensor data using the first set of sensors.

At block 906, a docking event is detected. Detecting a docking event can occur as disclosed herein, such as via detecting power being supplied from the docking device to the wearable device. In some cases, detecting a docking event can include i) detecting a physical connection (e.g., via a magnetic switch, a presence detector, a weight change, an impedance change, a capacitance change, a resistance change, an inductance change, a physical switch, etc.); ii) detecting a power connection; iii) detecting a data connection; or iv) any combination of i-iii.

In some cases, at optional block 908, capability information associated with the docking station can be determined. In such cases, capability information can be determined by receiving the capability information from the docking station (e.g., capability information stored on the docking station and transferred to the wearable device via a data connection), receiving the capability information manually (e.g., via user input), or by determining identification information associated with the docking station and using the identification information to look up the capability information. The capability information can indicate what sensor(s) and/or sensing parameters are available for use.

At block 910, the wearable device can be operated in a second mode. Operating the wearable device in a second mode can include receiving second sensor data at block 912. Receiving second sensor data at block 912 can include using a second sensor configuration that is different from the first sensor configuration of block 904. The second sensor configuration can be a predetermined sensor configuration or can be based at least in part on the determined capability information of block 908. Receiving second sensor data using the second sensor configuration can include collecting sensor data using one or more sensors of the wearable device and/or one or more sensors of the docking device. For example, sensor data collected by the docking device can be received by the wearable device via a data connection with the docking device. In some cases, the data connection can be used to provide data from the wearable device to the docking device, which can enable the docking device to handle data processing tasks, display results or other information, or otherwise make use of data from the wearable device.

In some cases, at optional block 914, first sensor data and/or second sensor data can be calibrated. Calibrating sensor data can include comparing the first sensor data and the second sensor data (e.g., comparing physiological parameters determined using the first sensor data and physiological parameters determined using the second sensor data) to determine whether adjustments to the first sensor data or second sensor data are needed to achieve the results expected based on the other of the first sensor data and second sensor data. For example, first sensor data can be adjusted until a given physiological parameter determined using the first sensor data matches the given physiological parameter determined using the second sensor data.

At block 916, a physiological parameter can be determined using the first sensor data and the second sensor data.

In some cases, at optional block 918, the wearable device can be operated in a third mode to receive third sensor data using a third sensor configuration that is different than the first sensor configuration and the second sensor configuration. In some cases, operating the wearable device in a third mode can include operating the wearable device in a power-saving mode, in which case the third sensor data is associated a third sensor configuration designed to conserve power. Operating the wearable device in such a mode can be automatically performed in response to receiving a low power signal.

In some cases, operating the wearable device in a third mode at block 918 can include operating the wearable device in a particular mode associated with a given sleep state, a given sleep stage, or a given sleep event. In such cases, operating the wearable device in the third mode can be in response to detecting a change in sleep state, detecting a change in sleep stage, or detecting a sleep event (e.g., an apnea). In such cases, the third sensor data can be based on a third sensor configuration designed to acquire certain data using a higher resolution, higher sampling rate, or otherwise improved.

In some cases, when third sensor data is received at block 918, calibrating that occurs at block 914 can include calibrating the third sensor data and/or calibrating first and/or second sensor data using the third sensor data.

While the blocks of process 900 are depicted in a certain order, some blocks can be removed, new blocks can be added, and/or blocks can be moved around and performed in other orders, as appropriate.

Various aspects of the present disclosure, such as those described with reference to process 900, can be performed by a wearable device, a docking device, a remote server (e.g., a cloud server), a user device (e.g., a smartphone or smartphone app), or any combination thereof. For example, receiving sensor data can include receiving sensor data at a wearable device, receiving sensor data at a docking device, receiving sensor data at a remote server, receiving sensor data at a user device, or any combination thereof.

One or more elements or aspects or steps, or any portion(s) thereof, from one or more of any of claims 1 to 43 below can be combined with one or more elements or aspects or steps, or any portion(s) thereof, from one or more of any of the other claims 1 to 43 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method, comprising:
operating a wearable device in a first mode, the wearable device having one or more sensors, wherein operating the wearable device in the first mode includes receiving first sensor data from at least one of the one or more sensors of the wearable device while the wearable device is being worn by a user;
detecting a docking event associated with coupling the wearable device to a docking device, wherein the wearable device receives power from the docking device when the wearable device is coupled with the docking device; and
automatically operating the wearable device in a second mode in response to detecting the docking event, wherein operating the wearable device in the second mode includes receiving second sensor data.

2. The method of claim 1, further comprising determining a physiological parameter associated with the user based at least in part on the first sensor data and the second sensor data.

3. The method of claim 1, wherein receiving the first sensor data includes operating the at least one of the one or more sensors according to a first set of sensing parameters, and wherein receiving the second sensor data includes operating the at least one of the one or more sensors according to a second set of sensing parameters that is different from the first set of sensing parameters.

4. The method of claim 3, wherein operating the at least one of the one or more sensors according to the first set of sensing parameters uses less power than operating the at least one of the one or more sensors according to the second set of sensing parameters.

5. The method of claim 3:
wherein the first set of sensing parameters includes i) a first sampling rate; ii) a first sampling depth; iii) a first gain; or iv) any combination of i-iii; and
wherein the second set of sensing parameters includes i) a second sampling rate that is greater than the first sampling rate; ii) a second sampling depth that is greater than the first sampling depth; iii) a second gain that is greater than the first gain; or iv) any combination of i-iii.

6. The method of claim 1, wherein receiving the second sensor data includes receiving the second sensor data from at least one or more additional sensors that are different from the at least one of the one or more sensors.

7. The method of claim 6, wherein the docking device includes at least one docking device sensor, and wherein the at least one docking device sensor includes the at least one or more additional sensors.

8. The method of claim 6, wherein the at least one of the one or more sensors includes a contacting sensor and wherein the at least one or more additional sensors includes a non-contacting sensor.

9. The method of claim 8, wherein the non-contacting sensor is an acoustic biomotion sensor.

10. The method of claim 6, wherein the at least one of the one or more sensors includes a line-of-sight sensor and wherein the at least one or more additional sensors includes a non-line-of-sight sensor.

11-13. (canceled)

14. The method of claim 1, further comprising transmitting the first sensor data and the second sensor data to the docking device.

15. The method of claim 1, wherein receiving the second sensor data includes receiving the second sensor data from the at least one of the one or more sensors and from at least one additional sensor.

16. The method of claim 1, wherein detecting the docking event includes detecting power being received by the wearable device.

17. The method of claim 1, wherein detecting the docking event includes confirming the docking event based at least in part on the first sensor data.

18-19. (canceled)

20. The method of claim 1, further comprising determining capability information associated with the docking station, wherein operating the wearable device in the second mode includes:
i) determining a sensing parameter based at least in part on the determined capability information;
ii) determining at least one additional sensor to use to receive second sensor data based at least in part on the determined capability information; or
iii) any combination of i or ii.

21-22. (canceled)

23. The method of claim 1, further comprising:
detecting a low power signal while the wearable device is being worn by the user; and
automatically operating the wearable device in a third mode, wherein operating the wearable device in the third mode includes receiving third sensor data using the at least one of the one or more sensors, and wherein operating the wearable device in the third mode uses less power than operating the wearable device in the first mode.

24. The method of claim 1, wherein the second sensor data is associated with the user engaging in a sleep session.

25. The method of claim 1, wherein operating the wearable device in the first mode occurs while the user is engaging in a first sleep session, and wherein operating the wearable device in the second mode occurs while the user is engaging in a second sleep session.

26-27. (canceled)

28. The method of claim 1, wherein receiving the second sensor data includes using the one or more sensors of the wearable device to collect the second sensor data, and wherein the docking device is shaped to facilitate collection of the second sensor data.

29-39. (canceled)

40. A system comprising:
a control system including one or more processors; and
a memory having stored thereon machine readable instructions, the memory being coupled to the control system;
wherein the machine executable instructions in the memory are executed by at least one of the one or more processors of the control system to
operate a wearable device in a first mode, the wearable device having one or more sensors, wherein operating the wearable device in the first mode includes receiving first sensor data from at least one of the one or more sensors of the wearable device while the wearable device is being worn by a user;
detect a docking event associated with coupling the wearable device to a docking device, wherein the wearable device receives power from the docking device when the wearable device is coupled with the docking device; and
automatically operate the wearable device in a second mode in response to detecting the docking event, wherein operating the wearable device in the second mode includes receiving second sensor data.

41-43. (canceled)
